(19) United States
(12) Patent Application Publication
(10) Pub. No.: US 2025/0352075 A1
(43) Pub. Date: Nov. 20, 2025

(54) ELECTRONIC DEVICE AND METHOD FOR DETERMINING A BODY CORE TEMPERATURE

(71) Applicant: Samsung Electronics Co., Ltd., Suwon-si (KR)

(72) Inventors: Elena Konstantinovna VOLKOVA, Saratov (RU); Konstantin Alexandrovich PAVLOV, Zelenograd (RU); Kirill Gennadievich BELIAEV, St. Petersburg (RU); Alexey Vyacheslavovich PERCHIK, Moscow (RU); Vladislav Valerievich LYCHAGOV, Saratov (RU); Evgenii Alexandrovich NIKOLAEV, Cheboksary (RU); Alexey Anatolievich AYUEV, Kurgan (RU); Georgii Albertovich NIGMATULIN, Moscow (RU); Wonseok LEE, Suwon-si (KR); Hongji LEE, Suwon-si (KR); Younghyun KIM, Suwon-si (KR)

(21) Appl. No.: 19/285,600

(22) Filed: Jul. 30, 2025

Related U.S. Application Data
(63) Continuation of application No. PCT/KR2023/012505, filed on Aug. 23, 2023.

(30) Foreign Application Priority Data
Feb. 1, 2023 (RU) 2023102265

(51) Int. Cl. A61B 5/0205; A61B 5/00; A61B 5/024; A61B 5/0295; A61B 5/053

(52) U.S. Cl. CPC A61B 5/02055; A61B 5/0295; A61B 5/053; A61B 5/681; A61B 5/7207; A61B 5/7253; A61B 5/02416; A61B 2560/0252

(57) ABSTRACT

A method performed by an electronic device for determining a body core temperature of a user is provided. The method includes obtaining, by the electronic device, an internal temperature inside the electronic device, a skin surface temperature of the user, at least one body impedance parameter of the user and at least two PPG signals with different wavelengths, obtaining, by the electronic device, a measurement data set including the internal temperature, the skin surface temperature, the at least one body impedance parameter of the user and the at least two PPG signals, determining, by the electronic device, microclimate parameters around the electronic device based on a local ambient temperature and a local temperature, determining, by the electronic device, body morphology parameters of the user based on profile data of the user and the at least one body impedance parameter of the user, determining, by the electronic device, peripheral microcirculation parameters of the user body based on perfusion parameters of the user body which are calculated from the at least two PPG signals with different wavelengths, and determining, by the electronic device, the body core temperature of the user based on at least one of the measurement data set, the microclimate parameters around the electronic device, the body morphology parameters of the user, or the peripheral microcirculation parameters of the user.

### CROSS-REFERENCE TO RELATED APPLICATION(S)

This application is a continuation application, claiming priority under 35 U.S.C. § 365(c), of an International application No. PCT/KR2023/012505, filed on Aug. 23, 2023, which is based on and claims the benefit of a Russian patent application number 2023102265, filed on Feb. 1, 2023, in the Korean Intellectual Property Office, the disclosure of which is incorporated by reference herein in its entirety.

### BACKGROUND

### 1. Field

The disclosure relates to devices, methods, and systems for non-invasive, personal and/or on-demand health monitoring. More particularly, the disclosure relates to the monitoring of body core temperature. In addition, the disclosure relates to development of machine learning algorithms for accurately determining the body core temperature based on data collected from sensors used in electronic devices, such as modern smart watches, smart rings and fitness bracelets.

### 2. Description of Related Art

Measurement of a body temperature plays an important role in monitoring vital signs of human health in everyday life (at work, at home, during sport activities). This is due to the fact that many diseases are accompanied by characteristic changes in the body temperature. Temperature monitoring makes it possible to reveal a disease at an early stage. The advantage of the body temperature measurement using electronic devices, in particular smart watches, smart rings or fitness bracelets, is a non-invasive measurement method, fast measurement, ease of use, the ability to measure in the dark, the ability of continuous and long-term temperature monitoring.

However, modern electronic devices with a function of tracking temperature, such as smart watches, smart rings and fitness bracelets, are designed to measure a body surface temperature rather than a body core temperature, which is commonly used in medical practice.

In the scientific and medical literature, there are two main zones for measuring a temperature of a human body—a core and a shell, where the core is understood as blood and all internal organs, and the shell is understood as the skin and other superficially located structures. The core is a part of the body that has a constant temperature, and the shell is a part of the body in which there is a temperature gradient, through which heat exchange occurs between the core and the environment. The temperature diagram of a person in cold and warm conditions is shown in FIG. 1A.

FIG. 1A illustrates the temperature scheme of a person in cold and warm conditions according to the related art.

For medical diagnostics, mercury or electronic medical thermometers are used, which are placed in the armpit, in the oral cavity, in the rectum, in the ear, etc., depending on standards adopted in a particular country. The place of measurement is one of the controversial and discussed issues in medicine, since there is a difference in temperature values of organs and tissues located superficially or in core regions of the human body.

The temperature of the body surface is in different temperature ranges compared to the body core temperature characteristic of the core, since it is influenced by a number of internal and external factors, due to which the accuracy of temperature measurements using electronic devices located, for example, in the limbs (wrist, finger, etc.) may differ markedly from the results acquired with medical thermometers.

Among the main external factors affecting the skin surface temperature measured by an electronic device, one can list the surface area on which the measurement takes place, a part of the body on which the measurement takes place, the presence of contact between the device and the user skin, etc. Also, the accuracy of determining the surface temperature skin is influenced by the parameters of the environment in which the user is located, in particular, ambient temperature, humidity, air speed (wind), the presence of clothing on one or another part of the human body where an electronic device is located (for example, a person is in the cold without mittens) and etc.

The internal factors affecting the skin surface temperature measured by an electronic device include the morphology (composition) of the human body and the peripheral microcirculation of the human body.

Human skin and fat are known to have lower thermal conductivity than other major organs and tissues, which allows the human body to retain heat. Knowing the morphological parameters that define an amount of a fat tissue, a muscle tissue, water in the human body, a ratio of extracellular and intracellular water in the human body, a state of human skin (wet or dry), one can evaluate their effect on heat transfer through the shell and take this effect into account when determining body core temperature based on the skin surface temperature measured by the electronic device.

Since the main part of the heat transferred from the core of the body to the surface of the skin comes from the vessels, it is also very important to take into account peripheral microcirculation parameters of the human body. For example, there are problems such as impaired peripheral microcirculation when a person has constantly cold hands or when a person has constantly hot hands (FIG. 1B).

FIG. 1B illustrates the change in the temperature scheme of a person with violations of the peripheral blood microcirculation (constantly cold hands or constantly hot hands) according to the related art.

Without taking these factors into account, an error may occur when determining the body core temperature based on the skin surface temperature measured by an electronic device located on the arm.

US20220171344A1 discloses an electronic device comprising at least one photoplethysmogram (PPG) sensor and ambient and body temperature sensors.

The disadvantages of this technical solution are related to the fact that it lacks information describing temperature measurements. In addition, US20220171344A1 does not use additional data such as peripheral microcirculation parameters or body morphology parameters of a human when determining a body temperature.

US20210290072A1 describes an electronic device for a noninvasive measurement of a body temperature, comprising one or more temperature sensors for measuring temperature data of a subject, and an impedance sensor. Also, the electronic device has a thermally conductive probe located in an opening of the device and configured to transfer thermal energy from the subject to said one or more temperature sensors when the electronic device is attached to a user. By insulating a skin surface in the opening around the probe, heat leakage is prevented, so a temperature gradient between the core of the body and the skin surface is reduced. The temperature of the skin under the isolated area rises until it reaches equilibrium with the warmest area (i.e., the core of the body), thereby approaching the temperature of the core of the body.

The disadvantages of this technical solution are related to the fact that additional data, such as environmental parameters, body peripheral microcirculation parameters, or body morphology parameters of a human, are not used when determining the body core temperature. In addition, a longer measurement time is required to achieve thermal equilibrium.

U.S. Ser. No. 11/224,344B2 describes a method and system for determining a body core temperature. The system contains pairs of temperature sensors thermally connected to different heat transfer channels associated with different measurement locations, i.e. various external areas of the human skin. The system is also configured to determine the perfusion parameter and ambient temperature as additional parameters.

The disadvantages of this technical solution are related to the fact that body morphology parameters are not used when assessing the body core temperature. In addition, direct measurements of perfusion parameters are not used in this document, as perfusion parameters are retrieved based on temperature data.

Thus, there is a need to develop devices, methods and systems with a function of determining the core temperature of the user body in real time, capable of taking into account a large number of factors that affect the transfer of heat from the core of the body to the surface of a human skin: an air temperature; air humidity; air blowing speed; thermal conductivity of skin tissues; convection of systemic circulation; convection of the skin blood flow; intensity of metabolic processes in underlying tissues, physical activity and time of day.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an electronic device and method for determining a body core temperature.

Additional aspects will be set forth in part in the description which follows and, in part, will be apparent from the description, or may be learned by practice of the presented embodiments.

In accordance with an aspect of the disclosure, a method performed by an electronic device for determining a body core temperature of a user is provided. The method includes obtaining, by the electronic device, an internal temperature inside the electronic device, a skin surface temperature of the user, at least one body impedance parameter of the user and at least two PPG signals with different wavelengths, obtaining, by the electronic device, a measurement data set including the internal temperature, the skin surface temperature, the at least one body impedance parameter of the user and the at least two PPG signals, determining, by the electronic device, microclimate parameters around the electronic device based on a local ambient temperature and a local temperature; determining, by the electronic device, body morphology parameters of the user based on profile data of the user and the at least one body impedance parameter of the user, determining, by the electronic device, peripheral microcirculation parameters of the user body based on perfusion parameters of the user body which are calculated from the at least two PPG signals with different wavelengths, and determining, by the electronic device, the body core temperature of the user based on at least one of the measurement data set, the microclimate parameters around the electronic device, the body morphology parameters of the user, or the peripheral microcirculation parameters of the user.

In accordance with another aspect of the disclosure, an electronic device for determining a body core temperature of a user is provided. The electronic device includes at least one internal temperature sensor configured to measure an internal temperature inside the electronic device, at least one skin temperature sensor configured to measure a skin surface temperature of the user, at least one bioimpedance sensor configured to measure at least one body impedance parameter of the user, at least one photoplethysmogram (PPG) sensor configured to measure at least two photoplethysmogram (PPG) signals with different wavelengths, memory, comprising one or more storage media, storing instructions, and at least one processor communicatively coupled to the at least one internal temperature sensor, the at least one skin temperature sensor, the at least one bioimpedance sensor, the at least one PPG sensor, and the memory, wherein the instructions, when executed by the at least one processor individually or collectively, cause the electronic device to obtain the internal temperature inside the electronic device, the skin surface temperature of the user, the at least one body impedance parameter of the user and the at least two PPG signals with different wavelengths, obtain a measurement data set including the internal temperature, the skin surface temperature, the at least one body impedance parameter of the user and the at least two PPG signals, determine microclimate parameters around the electronic device based on a local ambient temperature and a local temperature, determine body morphology parameters of the user based on profile data of the user and the at least one body impedance parameter of the user, determine peripheral microcirculation parameters of the user body based on perfusion parameters of the user body which are calculated from the at least two PPG signals with different wavelengths, and determine the body core temperature of the user based on at least one of the measurement data set, the microclimate parameters around the electronic device, the body morphology parameters of the user, or the peripheral microcirculation parameters of the user.

In accordance with another aspect of the disclosure, one or more non-transitory computer-readable storage media storing one or more computer programs including computer-executable instructions that, when executed by at least one processor of an electronic device individually or collectively, cause the electronic device to perform operations are provided. The operations include obtaining, by the electronic device, an internal temperature inside the electronic device, a skin surface temperature of a user, at least one body impedance parameter of the user and at least two PPG signals with different wavelengths, obtaining, by the electronic device, a measurement data set including the internal temperature, the skin surface temperature, the at least one body impedance parameter of the user and the at least two PPG signals, determining, by the electronic device, microclimate parameters around the electronic device based on a local ambient temperature and a local temperature, determining, by the electronic device, body morphology parameters of the user based on profile data of the user and the at least one body impedance parameter of the user, determining, by the electronic device, peripheral microcirculation parameters of the user body based on perfusion parameters of the user body which are calculated from the at least two PPG signals with different wavelengths, and determining, by the electronic device, a body core temperature of the user based on at least one of the measurement data set, the microclimate parameters around the electronic device, the body morphology parameters of the user, or the peripheral microcirculation parameters of the user.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A illustrates the temperature scheme of a person in cold and warm conditions according to the related art;

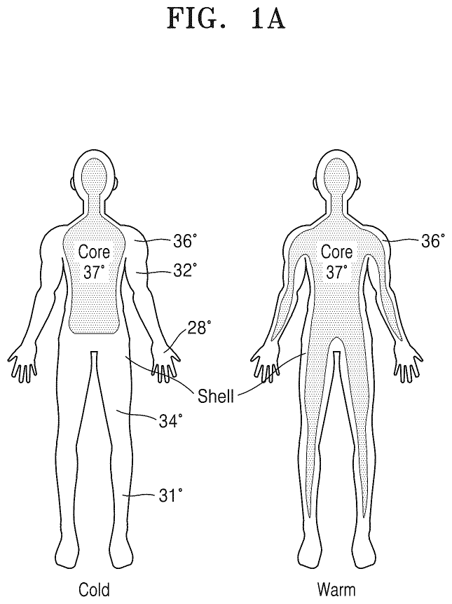

FIG. 1B illustrates the change in the temperature scheme of a person with violations of the peripheral blood microcirculation (constantly cold hands or constantly hot hands) according to the related art;

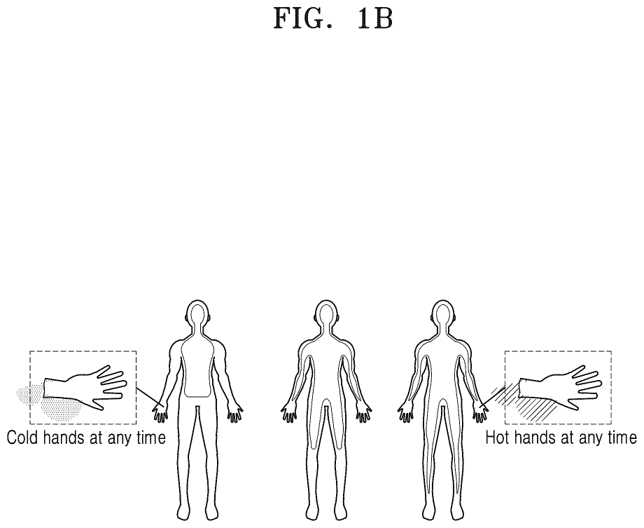

FIG. 2A illustrates a schematic representation of an electronic device with a function of determining a body core temperature of a person according to an embodiment of the disclosure;

FIG. 2B illustrates a top view and a bottom view of an embodiment of an electronic device in the form of a smart watch according to an embodiment of the disclosure;

FIG. 2C illustrates the location of at least one internal temperature sensor and at least one skin temperature sensor of an electronic device in the form of a smart-watch according to an embodiment of the disclosure;

FIG. 2D illustrates the location of electrodes of at least one bioimpedance of an electronic device in the form of the smart-watch according to an embodiment of the disclosure;

FIG. 2E illustrates the location of at least one PPG sensor of an electronic device in the form of a smart-watch according to an embodiment of the disclosure;

FIG. 3 illustrates the external and internal parameters of the electronic device-user body part system taken into account in the prediction algorithm according to an embodiment of the disclosure;

FIG. 4A illustrates external factors affecting the accuracy of recalculating a skin surface temperature to a body core temperature according to an embodiment of the disclosure;

FIG. 4B illustrates the use of the first external reference device (ERD₁) when determining the microclimate parameters according to an embodiment of the disclosure;

FIG. 4C illustrates the use of a second external reference device (ERD₂) when determining the microclimate parameters according to an embodiment of the disclosure;

FIG. 4D illustrates a climate chamber with a mock-up of an electronic device located in it according to an embodiment of the disclosure;

FIG. 4E illustrates the experimental data obtained on a mock-up of an electronic device located in a climate chamber according to an embodiment of the disclosure;

FIG. 4F illustrates the prediction results from experimental data obtained with the climate chamber according to an embodiment of the disclosure;

FIG. 5 illustrates the structure of a biological tissue in the form of a sequence of flat layers with different thermal parameters that affect the transfer of heat from blood vessels to the skin surface according to an embodiment of the disclosure;

FIG. 6A illustrates the human body as a conductive cylinder and the corresponding scheme for representing a human body composition in bioimpedance analysis according to an embodiment of the disclosure;

FIG. 6B illustrates the use of a third external reference device (ERD₃) in determining the body morphology parameters of the user according to an embodiment of the disclosure;

FIG. 6C illustrates the results of predicting morphology parameters using at least one bioimpedance sensor of an electronic device and using a third external reference device according to an embodiment of the disclosure;

FIG. 6D illustrates the results of predicting the body morphology parameters (composition) of a tested subject based on a clinical database containing information on 578 subjects according to an embodiment of the disclosure;

FIG. 7A illustrates the reasons for using PPG signals with different wavelengths when predicting peripheral microcirculation parameters according to an embodiment of the disclosure;

FIG. 7B illustrates the filtering of PPG signals from motion artifacts according to an embodiment of the disclosure;

FIG. 7C illustrates the separation of PPG signals into time-constant and non-time-constant components according to an embodiment of the disclosure;

FIG. 7D illustrates the separation of the PPG signal into separate time segments (PPG pulses) according to an embodiment of the disclosure;

FIG. 7E illustrates the transformation of the time-constant and non-time-constant components of the PPG time series into a frequency domain according to an embodiment of the disclosure;

FIG. 7F illustrates the use of the fourth external reference device (ERD₄) in predicting the body morphology parameters of the user according to an embodiment of the disclosure;

FIG. 7G illustrates the effect of a combination of perfusion scores and PPG waveform features on the accuracy of body core temperature prediction according to an embodiment of the disclosure;

FIG. 8A illustrates the results of predicting the body core temperature of the user from a clinical database containing information on 271 tests performed on 88 test subjects according to an embodiment of the disclosure;

FIG. 8B illustrates the results of predicting the body core temperature of the user from a clinical database containing information on 484 tests performed on 266 test subjects according to an embodiment of the disclosure;

FIG. 9A illustrates schematically the sequence of steps in the method of operation of an electronic device with a function of determining a body core temperature of the user according to an embodiment of the disclosure;

FIG. 9B illustrates the sub-steps of the prediction step performed in the method of operation of the electronic device with a function of determining the body core temperature of the user according to an embodiment of the disclosure;

FIG. 9C illustrates an example method for determining a body core temperature of the user according to an embodiment of the disclosure;

FIG. 10A illustrates a schematic representation of an embodiment of a system comprising an electronic device with a function of determining a body core temperature of a person according to an embodiment of the disclosure; and

FIG. 10B illustrates a schematic representation of an embodiment of a system comprising an electronic device with a function of determining a body core temperature of a person according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Further, skilled artisans will appreciate that elements in the drawings are illustrated for simplicity and may not have necessarily been drawn to scale. For example, the flowcharts illustrate the method in terms of the most prominent steps involved to help to improve understanding of aspects of the disclosure. Furthermore, in terms of the construction of the device, one or more components of the device may have been represented in the drawings by conventional symbols, and the drawings may show only those specific details that are pertinent to understanding the embodiments of the disclosure so as not to obscure the drawings with details that will be readily apparent to those of ordinary skill in the art having the benefit of the description herein.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or one embodiment or several embodiments or all embodiments. Accordingly, the term “some embodiments” is defined as meaning “no embodiment, or one embodiment, or more than one embodiment, or all embodiments.”

The terminology and structure employed herein are for describing, teaching, and illuminating some embodiments and their specific features and elements and do not limit, restrict, or reduce the spirit and scope of the claims or their equivalents.

More specifically, any terms used herein such as but not limited to “includes,” “comprises,” “has,” “consists,” and grammatical variants thereof do not specify an exact limitation or restriction and certainly do not exclude the possible addition of one or more features or elements, unless otherwise stated, and must not be taken to exclude the possible removal of one or more of the listed features and elements, unless otherwise stated.

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do not preclude there being none of that feature or element unless otherwise stated. Thus, at least one of A, B or C may be referred to as “only a”, “only b”, “only c”, “both a and b”, “both a and c”, “both b and c”, “all of a, b, and c”, or variations thereof.

Unless otherwise defined, all terms, and especially any technical and/or scientific terms, used herein may be taken to have the same meaning as commonly understood by one having ordinary skill in the art.

The disclosure has been made in view of a number of known solutions shown above, and is directed, in particular, to elimination and/or mitigation of at least some of the shortcomings of these known solutions.

In particular, some embodiments of the disclosure provide an electronic device with a function of determining a body core temperature of a user, comprising: at least one internal temperature sensor configured to measure the temperature inside the electronic device, at least one skin temperature sensor configured to measure a skin surface temperature of the user, at least one bioimpedance sensor configured to measure at least one body impedance parameter of the user, at least one photoplethysmogram (PPG) sensor configured to measure at least two photoplethysmogram (PPG) signals with different lengths waves, wherein the electronic device is configured to core temperature of the user based on at least a measurement data set collected from said sensors.

In addition, some embodiments of the disclosure provide a method for operating the electronic device, and a system comprising said electronic device.

Those skilled in the art will appreciate that the various embodiments of the disclosure should in no way be construed as defining or limiting the scope of the claimed disclosure, and that other material and technical means equivalent or clearly analogous to those listed below may be envisaged by those skilled in the art to accomplish various operations, functions, method steps and the like described below. The present detailed description is not intended to define or limit the scope of the claimed disclosure, which should be defined only by reference to the appended claims.

The term “core” as used herein means the blood and all internal organs. The core has a constant temperature.

The term “shell” as used herein includes skin and other superficial structures. There is a temperature gradient in the shell; heat exchange between the core and the environment takes place through it.

The term “body core temperature” as used herein means the temperature of the central nervous system and the internal organs of the chest and abdomen, which is 2-4 degrees higher than the temperature of the extremities. Also in the scientific literature, synonymous terms are used: “body core temperature”, “internal body temperature”. Normally, the core temperature of the human body varies from 36.0° C. to 37.5° C.

The term “shell temperature” as used herein means the temperature of the skin, subcutaneous fat, superficial muscles. Also in the scientific literature, the synonymous term “skin temperature” is used. The temperature of the skin in different parts of the human body within the comfortable temperature of the environment is: on the skin of the forehead 33.2° C.; on the chest 33.5° C.; on the hands 30.4° C., on the foot 26.5-27.0° C.

The term “body surface temperature” as used herein means the surface temperature of the skin. The skin temperature can be obtained by the temperature sensor of the electronic device.

The term “temperature scheme of a person” used herein means the individual distribution of temperature indicators over the surface of the skin and various organs. It is individual for each person and it is relatively constant under normal conditions.

The term “peripheral microcirculation” as used herein means the movement of blood through microvessels (capillaries).

The term “bioimpedancemetry”, as used herein, means an analysis performed with specialized equipment that measures the electrical resistance of body cells.

The term “bioimpedance analysis”, as used herein, means a contact method for measuring the electrical conductivity of biological tissues, which makes it possible to assess a wide range of morphological and physiological parameters of the body.

The term “impedance” as used herein refers to the electrical impedance to AC circuit current. For a biological object, the impedance has a composite (complex) character Z=(R,X), where R is an active ohmic component, X is a reactive capacitive component of impedance. Impedance is defined by a magnitude |Z|=\sqrt{R²+X²} and a phase angle tgφ=X/R. The region of contact between electrodes and biological tissue contributes to the capacitive component of the impedance as well. Contact impedance (electrode-tissue contact) takes into account the physical and chemical processes occurring in the area of electrode contact with biological tissue (for example, electrode polarization, skin condition (wet or dry), etc.).

The term “sub-algorithm” as used herein means an algorithm that is used entirely within an algorithm.

The term “computer-readable storage medium” as used herein means any means or group of means capable of storing data and/or instructions for some period of time. Computer-readable storage media may include, without limitation, storage media such as direct access storage (for example, a hard disk drive or floppy disk), sequential access storage (for example, a tape drive), compact disc, CD-ROM, DVD, random access memory (RAM), read only memory (ROM), electrically erasable programmable read only memory (EEPROM), and/or flash memory; as well as communication media such as wires, optical fibers and other electromagnetic and/or optical media; and/or any combination of the aforementioned devices.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

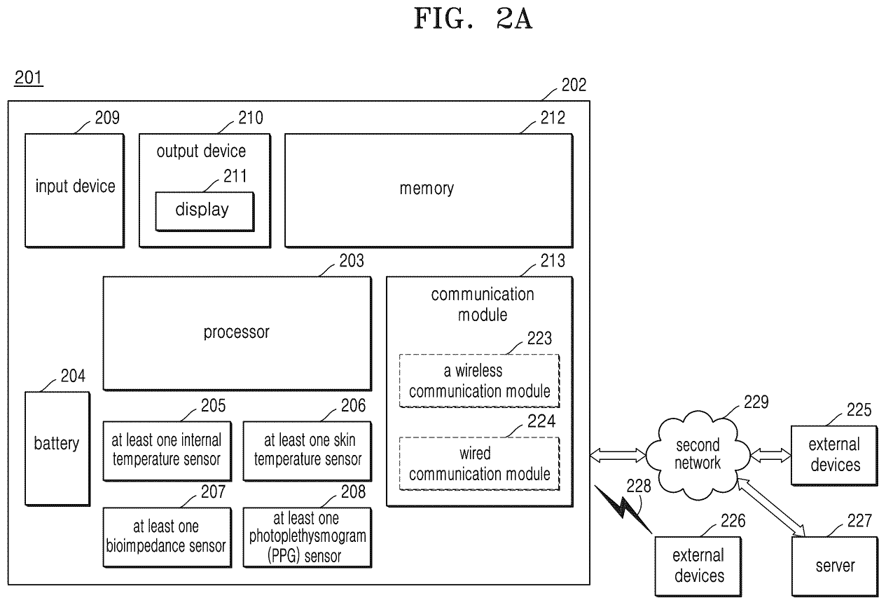

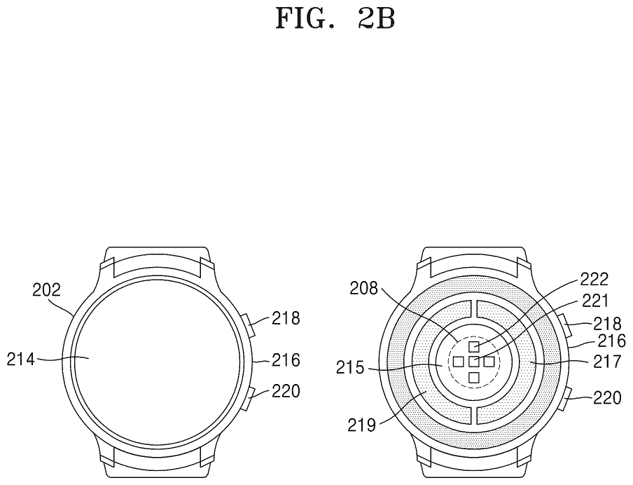

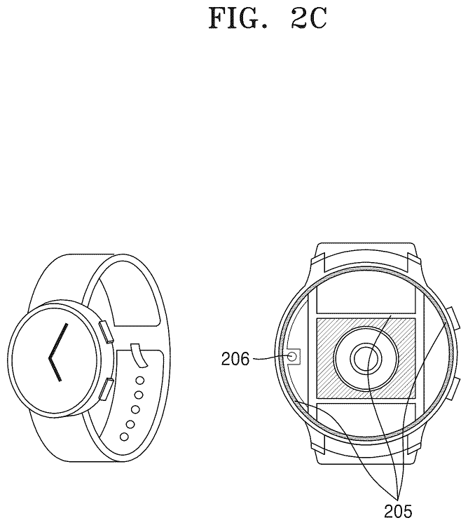

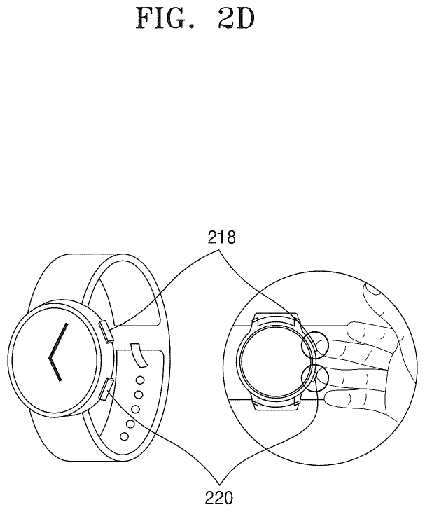

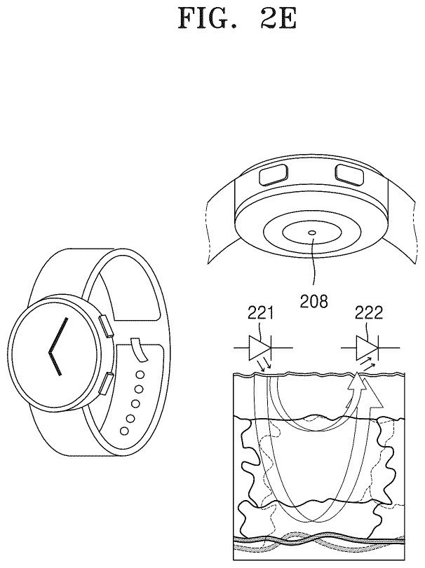

Next, FIGS. 2A to 2E show a schematic representation of an electronic device 201 with a function of determining the body core temperature of a person according to various embodiments of the disclosure.

Referring to FIG. 2A, an embodiment of an electronic device 201 will be described comprising a housing 202, at least one processor 203, a battery 204, at least one internal temperature sensor 205, at least one skin temperature sensor 206, at least one bioimpedance sensor 207, at least one photoplethysmogram (PPG) sensor 208, an input device 209, an output device 210, a display 211, memory 212, and a communication module 213 including a wireless communication module 223 and a wired communication module 224. The electronic device 201 may include additional sensors, such as inertial motion sensors, gyroscopes, accelerometers, an electrocardiogram (ECG) measurement sensor, an atmospheric pressure sensor, a humidity sensor, a hall sensor, an ambient light sensor, etc. In some embodiments, the electronic device 201 may be a smart device such as a smart watch, smart ring or fitness bracelets, a medical electronic health monitoring device with a function of determining a body core temperature, etc. The electronic device is an electronic device. It can be configured to establish a wired or wireless communication channel with external devices (for example, devices 225, 226, such as smartphones, fitness bracelets, voice assistants, smart TVs, smart watches, etc.) or a server 227 and with the ability to transmit data through networks 228, 229.

Referring to FIGS. 2B to 2E, the electronic device 201 is in the form of a smartwatch that is worn on the wrist. In some embodiments of the disclosure (not shown), the electronic device 201 may be configured to be located on other parts of the user body (e.g., a finger).

FIG. 2B illustrates a top view and a bottom view of the electronic device 201 according to an embodiment of the disclosure. According to this embodiment, the housing 202 of the electronic device 201 may include a first surface 214 (or front surface), a second surface (or back surface) 215, and a side surface 216 surrounding the space between the first surface 214 and a second surface 215, and attachment elements connected to at least a portion of the housing 202 and configured to detachably attach the electronic device 201 to the wrist).

According to an embodiment of the disclosure, the at least one processor 203 can acquire temperatures inside the electronic device 201, for example, housing temperature, Tₕₒᵤₛ, processor temperature, T_{proc.}, and battery temperature, T_{bat.}, from the at least one internal temperature sensor 205, store them in memory 212 and load them from the memory 212 to perform operations. The at least one processor 203 may acquire the skin surface temperature of the user, T_{skin surf.}, from the at least one skin temperature sensor 206, store it in the memory 212, and load it from the memory 212 for operations. The at least one processor 203 may receive at least one impedance parameter from said at least one bioimpedance sensor 207, store it in the memory 212, and load it from the memory 212 to perform operations. The at least one processor 203 may receive said at least two PPG signals from said at least one PPG sensor 208, store them in the memory 212, and load them from the memory 212 to perform operations.

The at least one processor 203 can predict microclimate parameters around the electronic device, i.e. a local ambient temperature around the electronic device; and a local temperature between the electronic device and the user skin. Here, the at least one processor can predict the local ambient temperature, T_{loc.amb.}, around the electronic device based on the temperatures inside the electronic device, for example, T_{hous.}, T_{proc.}, T_{bat.}, obtained by said at least one internal temperature sensor 205, and then predict the local temperature between the electronic device and the user skin, T_{loc.device}, based on said local ambient temperature, T_{loc.amb.}, and the skin surface temperature of the user, T_{skin surf.}, obtained by said at least one skin temperature sensor 206.

The at least one processor 203 can predict the body morphology parameters of the user which define an amount of a fat tissue, a muscle tissue, water in the user body, a ratio of extracellular and intracellular water in the user body, and a condition of the user skin. The condition of the user may include thickness of the user skin or wetness of the user skin. This can be achieved, for example, based on the profile data of the user which are pre-entered into the electronic device and said at least one body impedance parameter of the user which is obtained by said at least one bioimpedance sensor 207.

The at least one processor can predict the peripheral microcirculation parameters of the user based on the body perfusion parameters (perfusion index (PI) of the user, PPG waveform features) which are calculated from said at least two PPG signals with different wavelengths captured by said at least one PPG sensor 208. In some embodiments of the disclosure, at least one processor 203 may filter said at least two PPG signals with different wavelengths from motion artifacts, separate said at least two PPG signals with different wavelengths into time-constant and non-time-constant time series components, and transform these time series into a frequency domain or a time-frequency domain and then extracting their amplitude-phase harmonic characteristics, where the transformation of said time series into the frequency domain may comprise a Fourier transform or a Hilbert-Huang transform, and the conversion of said time series into the frequency-time domain may comprise a Wavelet transform.

The at least one processor 203 may predict the body core temperature of the user, T_{core} based on at least said parameters of the microclimate around the electronic device, said body morphology parameters of the user, and said parameters of the body peripheral microcirculation of the user.

The at least one processor 203 may receive additional data (e.g., data from said additional sensors of the electronic device 201), store it in the memory 212 of the electronic device 201, and also load it from the memory 212 to perform operations. The at least one processor 203 may store the data obtained when predicting said parameters of the microclimate around the electronic device, the body morphology parameters of the user and the peripheral microcirculation parameters of the user into the memory 212 of the electronic device 201, and also load it from the memory 212 to perform operations.

The at least one processor 203 of the electronic device 201 may transmit information about the body core temperature of the user, determined based on the readings of said sensors 205, 206, 207, 208, to the output device 210 to inform the user.

The at least one processor 203 may download data received from the communication module 213 to the memory 212 and/or download data from the memory 212 to the communication module 213 for transmission to an external device (e.g., device 225, device 226, server 227, or cloud storage).

The battery 204 can supply power to at least one component of the electronic device 201.

The at least one internal temperature sensor 205 may be configured to measure the temperature inside the electronic device 201. The at least one internal temperature sensor 205 may be a resistive temperature sensor. In an embodiment of the disclosure shown in FIG. 2B, the electronic device 201 may include three internal temperature sensors 205 to measure the temperature of housing 202, Tₕₒᵤₛ, the temperature of processor 203, T_{proc}, the temperature of battery 204, T_{bat}. In an embodiment of the disclosure (not shown) only one internal temperature sensor 205 can be used in the electronic device 201 to measure the temperature of housing 202, Thus. In an embodiment of the disclosure (not shown), the electronic device 201 may use two internal temperature sensors 205, one of which measures, for example, the temperature of housing 202 and the other one measures the temperature of the battery 204. In an embodiment of the disclosure, the electronic device 201 can provide measurement of the temperatures of other internal components.

Said at least one skin temperature sensor 206 may be configured to measure the skin surface temperature of the user, T_{skin surf.}. It can be made in the form of an IR temperature sensor or a resistive temperature sensor. Said at least one skin temperature sensor 206 may be located within the part of the housing 202 facing the skin of the user, and in the case of its implementation in the form of a resistive sensor, it shall also be in thermal contact with the skin of the user. In the embodiment of the disclosure shown in FIG. 2B, the electronic device 201 includes one IR skin temperature sensor 206. In some embodiments of the disclosure, the electronic device may be provided with several skin temperature sensors 206. In this case, readings from the several skin temperature sensors 206 may be summed and averaged to obtain T_{skin surf.}.

Said at least one bioimpedance sensor 207 may be configured to measure at least one body impedance parameter of the user, wherein said at least one body impedance parameter of the user includes a magnitude of body impedance of the user, a phase angle of body impedance of the user, and a magnitude of a contact impedance. In the embodiment of the disclosure shown in FIGS. 2B and 2D, the electronic device 201 includes one bioimpedance sensor 207 for measuring at least one body impedance parameter of the user.

Referring to FIGS. 2B and 2D, it is shown that said at least one bioimpedance sensor 207 may comprise four electrodes: a first electrode 217, a second electrode 218, a third electrode 219 and a fourth electrode 220. In an embodiment of the disclosure, the second surface 215 may include a first electrode 217 and a third electrode 219, wherein the side surface 216 may include a second electrode 218 and a fourth electrode 220. In an embodiment of the disclosure (not shown), several PPG sensors may be provided in the electronic device 201.

The circuit for measuring said at least one impedance parameter used in the disclosure may be similar to the measurement circuit detailed in WO 2020138667 A1. Such a measurement circuit makes it possible to evaluate both a pure impedance of a body (without the influence of electrode contacts with the skin), and to evaluate a contact impedance separately (only impedances of the electrode contacts with the skin).

In the embodiment of the disclosure shown in FIGS. 2B and 2D, the user wears the electronic device 201 on the wrist such that the first electrode 217 and the third electrode 219 are in contact with the wrist and makes contact with the second electrode 218 and the fourth electrode 220 with the fingers (see FIG. 2D).

Said at least one photoplethysmogram (PPG) sensor 208 may be configured to measure at least two photoplethysmogram (PPG) signals with different wavelengths. In the embodiment of the disclosure shown in FIGS. 2B and 2E, said at least one PPG sensor 208 may be located on the side of the second surface 215 of the housing 202 (i.e., the surface facing the user skin). In an embodiment of the disclosure (not shown), the electronic device 201 may comprise two PPG sensors 208, each containing one radiation source 221 and one radiation receiver 222, wherein the radiation sources 221 of these two PPG sensors have different wavelengths. In this embodiment of the disclosure, two PPG sensors 208 will be able to capture 2 PPG signals with different wavelengths. In the embodiment of the disclosure shown in FIG. 2B, the electronic device 201 may include one PPG sensor 208, which may include, for example, two radiation sources 221 and, for example, two radiation receivers 222, wherein said radiation sources 221 having different wavelengths. In this embodiment of the disclosure, one PPG sensor 208 will be able to capture 2 PPG signals with different wavelengths. In an embodiment of the disclosure, the radiation source 221 may comprise a light emitting diode (LED).

The input device 209 may receive data, entered from outside (e.g., by a user of the electronic device 201), that can be used by a component (e.g., at least one processor 203) of the electronic device 201. In an embodiment of the disclosure, the input device 209 may be configured to input a profile data of the user containing a gender, an age, a height, a weight of the user. The at least one processor 203 of the electronic device 201 may receive the profile data of the user from the input device 209, store the profile data of the user in the memory 212, read it from the memory 212, and use it when predicting the body morphology parameters of the user. Also, the electronic device 201 may be provided for voice input and data output. Additionally, input and output of data can be performed by connecting to the Internet.

The output device 210 may be configured to output information to a user on the first side 214 of the electronic device 201. The output device 210 may include a display 211 that may be configured to display output information and located on the first side 214 of the electronic device 201. Additionally, the display 211 may be configured to use an on-screen keyboard to enter data such as a profile data of the user.

The memory 212 of the electronic device 201 may be configured to store the following data:
the profile data of the user containing the gender, the age, the height, the weight of the user,
measurement data sets including:
   temperatures inside the electronic device, for example, T_{hous.}, T_{proc.}, T_{bat}, obtained by said at least one internal temperature sensor 205,
   the skin surface temperature of the user, Tₛₖᵢₙ, obtained by said at least one skin temperature sensor 206,
   at least one body impedance parameter of the user which is obtained by said at least one bioimpedance sensor 207,
   at least two PPG signals with different wavelengths obtained by said at least one PPG sensor 208,
dates of collection of each of measurement data set;
times of collection of each of measurement data set,
body core temperatures of the user, T_{core} determined by the at least one processor for each of measurement data set, etc.

Also, the memory 212 can store various additional data, for example, data used and calculated by the at least one processor 203 of the electronic device 201 when determining the body core temperature of the user, in particular, parameters of microclimate around the electronic device, body morphology parameters of the user, peripheral microcirculation parameters of the user, or data taken from additional sensors of the electronic device 201.

The memory 212 may store various instructions that, when executed by the at least one processor 203, cause the at least one processor 203 to control the components of the electronic device 201 coupled to the at least one processor 203 and perform various data processing or calculations.

The communication module 213 may include a wireless communication module 223 (e.g., a cellular communication module, a short-range wireless communication module, or a global navigation satellite system (GNSS) communication module) or a wired communication module 224 (e.g., a local area network (LAN) communication module or power line communication module (PLC)). The communication module 213 may support establishing a wireless communication channel between the electronic device 201 and an external device (e.g., device 225, device 226, server 227, or a cloud storage) and communicating through the established communication channel. The communication module 213 may include one or more communication processors that operate independently of the at least one processor 203 and support direct (e.g., wired) communication or wireless communication. A respective one of these communication modules may communicate with an external electronic device via the first network 228 (e.g., a short-range communication network such as Bluetooth™, wireless (Wi-Fi), or infrared data (IrDA)) or the second network 229 (e.g., a long distance communication network such as a cellular network, the Internet, or a computer network (e.g., a local area network or a wide area network (WAN)).

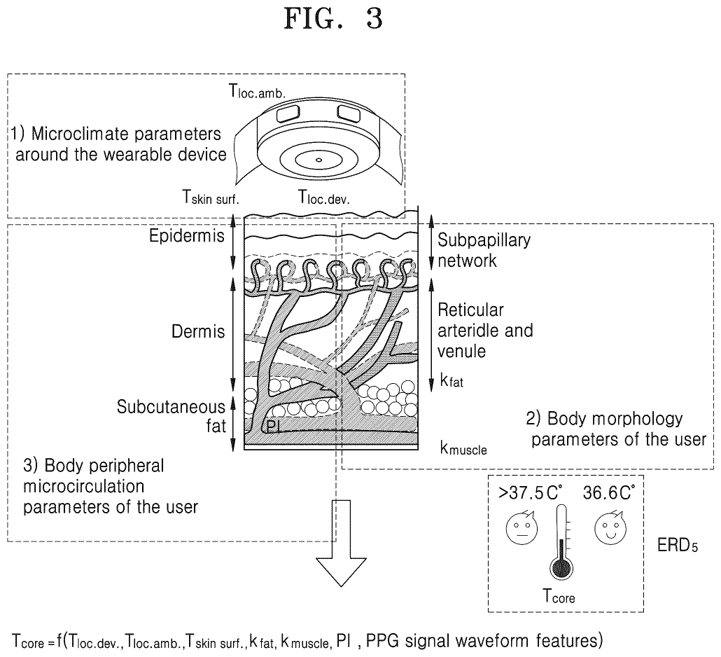

Next, with reference to FIGS. 3, 4A to 4F, 5, 6A to 6D, and 7A to 7G, determination of a body core temperature by prediction based on at least the readings of said sensors 205, 206, 207, 208 will be described.

FIG. 3 illustrates the external and internal parameters of the electronic device-user body part system taken into account in the prediction according to an embodiment of the disclosure.

Referring to FIG. 3, determining of the body core temperature of the user includes:
1) prediction of the microclimate parameters around the electronic device,
2) prediction of the body morphology parameters (composition) of the user,
3) predicting the peripheral microcirculation parameters of the user,
4) predicting the body core temperature of the user, where T_{core}=f(T_{loc.device}, T_{loc.amb.}, T_{skin surf.}, k_{fat}, k_{muscle}, PI, PPG signal waveform features).

### 1) Determining the Local Microclimate Parameters Around the Electronic Device

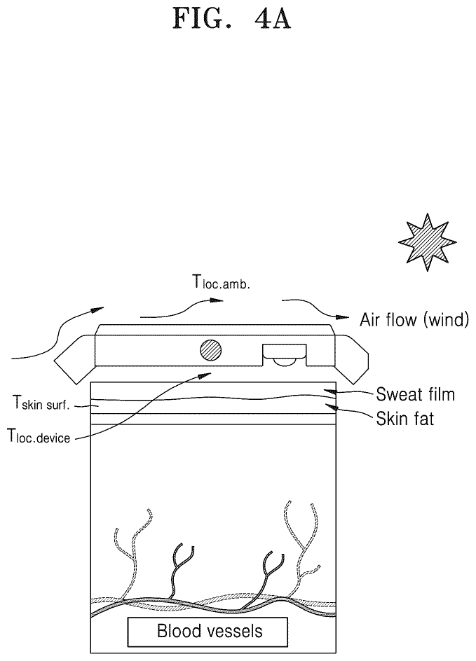

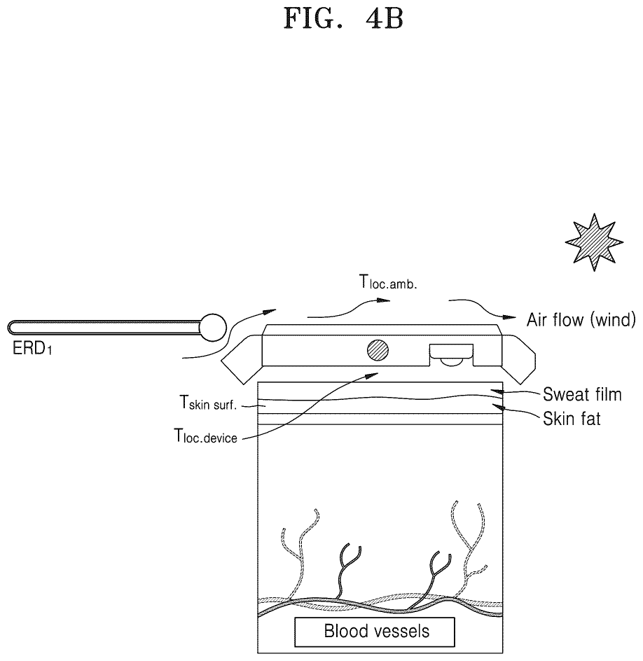

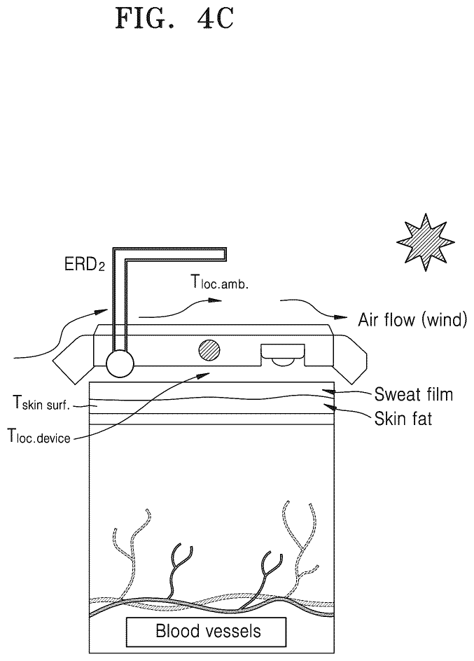

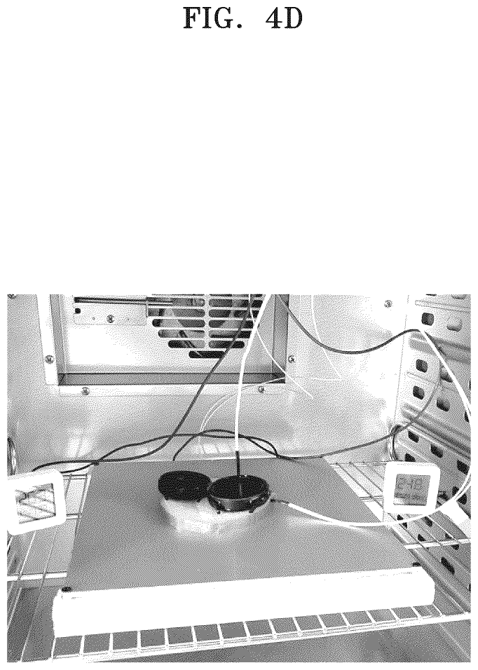

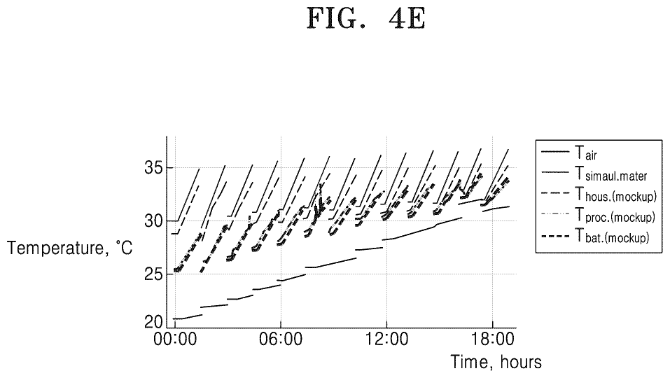

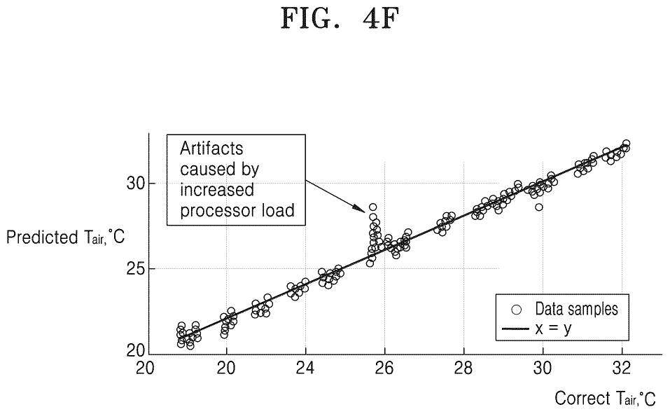

FIG. 4A illustrates external factors affecting the accuracy of recalculating a skin surface temperature to a body core temperature according to an embodiment of the disclosure.

It is shown with reference to FIG. 4A that around the electronic device-user body part system (for example, around the smart watch-wrist system) there can be a large number of external factors that affect the accuracy of recalculating the skin surface temperature to the body core temperature of the user. For example, an air flow may occur around the electronic device-user body part system, the electronic device may not fit snugly against the user body. When a person wears an electronic device for a long time (for example, he will wear a smart watch almost constantly), then such unpleasant moments arise as the formation of a film of sweat and a fatty film under the electronic device, which also affect the accuracy of recalculating the skin surface temperature to the body core temperature.

In order to take into account such factors, the disclosure provides for a local temperature prediction around the electronic device-user body part system, T_{loc.amb.}, and a local temperature prediction between the electronic device and the user skin, T_{loc.device}, which allows for a more accurate recalculation of a skin surface temperature, T_{skin surf.}, to a body core temperature of the user.

In the disclosure, this can be achieved by a first prediction sub-algorithm configured to predict the local ambient temperature, T_{loc.amb.}, around the electronic device based on temperatures inside the electronic device, e.g., T_{hous.}, T_{proc.}, T_{bat.}, obtained by at least one internal temperature sensor 205, and then predicting the local temperature between the electronic device and the user skin, T_{loc.device}, based on said local ambient temperature, T_{loc.amb.}, and the surface temperature of the user skin, T_{skin surf.}, obtained by at least one skin temperature sensor 206.

The first prediction sub-algorithm was created in advance as a result of neural network machine learning.

To train the first sub-algorithm for predicting the ability to predict the local microclimate parameters, the first database of training data was formed. To do this, a first plurality of other users (test subjects) were recruited, who wore an electronic device (its test sample) having at least one internal temperature sensor 205 on their wrist. For each of said first plurality of other users, readings were taken from said at least one internal temperature sensor 205, such as processor, housing, and battery temperatures.

Additionally, a first external reference device (ERD₁), such as a thermocouple, was used, which, for each of said first plurality of other users, made it possible to accurately determine the first reference temperature, T_{1 ref}, directly at the electronic device-user body part system. This first external reference device (ERD₁) is shown schematically as a circle in FIG. 4B.

FIG. 4B illustrates the use of the first external reference device (ERD₁) when determining the microclimate parameters according to an embodiment of the disclosure.

Temperatures inside the electronic device, e.g., T_{hous.}, T_{proc.}, T_{bat}, obtained by said at least one internal temperature sensor 205 for each of said plurality of other users, and a reference temperature, T_{1 ref.}, determined by the first external reference device (ERD₁) for each of the above plurality of users were allocated in the first training database.

The trained first prediction sub-algorithm made it possible to determine the temperature around the electronic device, T_{loc.amb.}, based on the temperatures inside the electronic device, for example, T_{hous.}, T_{proc.}, T_{bat}. After that, the temperature around the electronic device, T_{loc.amb.}, was also allocated in the first training database.

Next, for each of said first plurality of other users with the electronic device on the wrist, the skin surface temperature, T_{skin surf.}, was obtained by said at least one skin temperature sensor 206. Additionally, a second external reference device (ERD₂), such as a thermocouple, was used, which was located between the electronic device and the user body part and allowed to accurately determine the second reference temperature, T₂ ref, at this place. This second external reference device (ERD₂) is shown schematically as a circle in FIG. 4B.

Skin surface temperature, T_{skin surf.}, obtained by said at least one skin temperature sensor 206 for each of said first plurality of other users, and a second reference temperature, T_{2 ref}, obtained by the second external reference device (ERD₂) for each of said plurality of users, were also allocated in the first training database.

The first training data base supplemented in this way was used for further machine learning of the first prediction sub-algorithm. The additionally trained first prediction sub-algorithm was able to determine the local temperature between the electronic device and the user skin surface, T_{loc.device}, based on the temperature around the electronic device, T_{loc.amb.}, and the skin surface temperature, T_{skin surf.}. Thereafter, the temperature between the electronic device and the user skin surface, T_{loc.device}, was also allocated in the first training database. Thus, the first training data base was formed.

The solution to the problem of predicting the temperature around the electronic device and the local temperature between the electronic device and the skin surface was based on reveling empirical patterns in the training data allocated in the first database by machine.

FIG. 4C illustrates the use of a second external reference device (ERD₂) when determining the microclimate parameters according to an embodiment of the disclosure.

FIG. 4D illustrates a climate chamber with a mock-up of an electronic device located in it according to an embodiment of the disclosure.

It should be noted that the creation of the first prediction sub-algorithm based on the data of the test subjects collected in the first training database was preceded by experiments on a “non-living” laboratory setup that simulates an electronic device-user body part system. Measurements were taken on a mock-up of an electronic device placed in a climate chamber, as shown in FIG. 4D. In the climatic chamber, the mock-up of an electronic device was placed on a piece of material that imitated the thermophysical properties of a part of the user body. A piece of heat-treated pork was used as such a material. The climatic chamber maintained the set temperature within one degree and rebuilt it in time according to the set program. In the climate chamber, a fragment of material simulating the thermophysical properties of a part of the user body was fixed on a thermal table. The thermostatic table was independently controlled and made it possible to set the temperature of an object placed on it (a piece of material simulating the thermophysical properties of a part of the user's body), which could be greater than or equal to the air (ambient) temperature set by the camera. By means of temperature sensors installed in the mock-up of an electronic device, a temperature sensor installed in a fragment of material simulating the thermophysical properties of a part of the user body and an external reference measuring instrument (thermocouple), the following readings were taken: Tₐᵢᵣ—air temperature in the climatic chamber, T_{simul.mater.} is the temperature of a material fragment simulating the thermophysical properties of a user body part, T_{hous.(mockup)} is the temperature of the mockup body of the electronic device, T_{proc.(mockup)} is the temperature of the electronic device mockup processor, T_{bat.(mockup)} is the battery temperature of the electronic device mockup.

FIG. 4E illustrates experimental data obtained during 19-hour measurements with 85 stabilized states of a fragment of a material that simulates the thermophysical properties of a user body part according to an embodiment of the disclosure. On the graph, the x-axis shows the time in hours, the y-axis shows the temperature in degrees Celsius (° C.). In the experiments, the phantom “skin” temperature varied from 29° C. to 37° C., while the air temperature in the climate chamber varied from 21° C. to 32° C.

Further, the “ridge regression” machine learning model and the method of evaluating the effectiveness—cross-validation were used. When processing the data from FIG. 4E were split into 13 sets of incremental lines and cross-validated without considering one line. At the same time, the following were taken into account as features: “T_{simul.mater}”—temperature of a fragment of a material simulating the thermophysical properties of a part of the user body, the temperature difference “T_{proc.(mockup)}−T_{simul.mater}”, “T_{bat. (mockup)}−Tₐᵢᵣ”, “T_{simul.mater}−T_{body (mockup)}” “T_{simul.mater}−T_{body (mockup)}”, as well as features that define the load on the at least one processor.

FIG. 4F illustrates the results of predicting the air temperature in the climatic chamber according to the experimental data from FIG. 4E according to an embodiment of the disclosure. On the graph, the x-axis plots the air temperature in the climatic chamber, obtained by an external reference device (thermocouple), the y-axis plots the air temperature in the climate chamber, predicted from the readings of the temperature sensors of the mock-up of the electronic device and the temperature sensor in a fragment of material that simulates the thermophysical properties of a body part user. The graph also shows artifacts caused by increased processor load.

The accuracy characteristics of the machine learning model are shown in Table 1, where the following notation is used:
(Mean Absolute Error (MAE) is a metric that measures the average sum of the absolute difference between the actual value and the predicted value,
Root Mean Squared Error (RMSE)—the root of the average squared error,
R2corr—proportion of the variance of the dependent variable explained by the considered dependence model, that is, the explanatory variables,
Bias—systematic error,
Error standard deviation (SD)—standard deviation of the error.

TABLE 1
Machine Learning Model Accuracy Characteristics

| | |
|---|---|
| MAE, ° C. | 0.20 |
| RMSE, ° C. | 0.99 |
| R2corr | 0.79 |
| Bias, ° C. | 0.28 |
| Error SD, ° C. | 0.342 |

Thus, this experiment clearly demonstrated that the air temperature in the climatic chamber can be predicted based on the readings of temperature sensors taken on a “non-living” laboratory facility that simulates an electronic device-user body part system with an accuracy of ±0.2° C. (MAE=0.2° C.) relative to the external reference device (thermocouple).

### 2) Determination of Body Morphology Parameters (Composition) of a Human

To accurately transform the skin surface temperature of the user into the body core temperature of the user, it is necessary to take into account not only the external factors to which the user body part with the electronic device is exposed, but also the processes of bioheat exchange occurring in this part of the body. Bioheat transfer depends on the thermal properties of the skin and underlying tissues, as well as on the blood content of the tissue. The classical Pennes equation, which takes into account the effect of blood flow and metabolic heat on the energy balance in the tissue, has received the widest distribution for assessing bioheat exchange processes:

$${ρ}_{tissue} {C}_{tissue} \frac{\partial {T}_{tissue}}{\partial t} = ∇ {k}_{tissue} ∇ {T}_{tissue κ} + {ρ}_{blood} {C}_{blood} {W}_{blood} ( {T}_{art} - {T}_{tissue} ) + {q}_{met} ,$$

where rₜᵢₛₛᵤₑ is tissue density, C tissue is tissue heat capacity, T tissue is tissue temperature, kₜᵢₛₛᵤₑ is tissue thermal conductivity, r_{blood} is blood density, C blood is blood heat capacity, W_{blood} is blood perfusion rate, Cₐᵣₜ is arterial blood temperature, qₘₑₜ is metabolic heat.

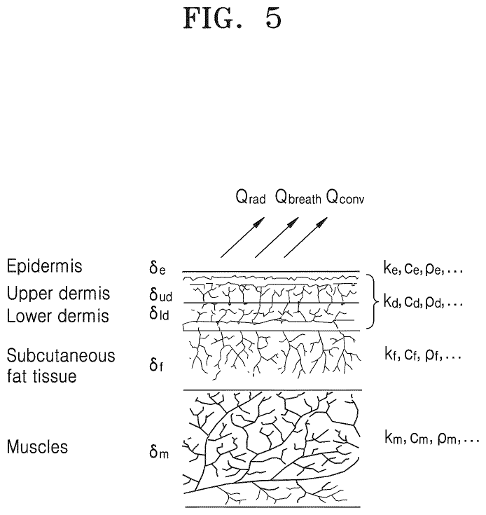

FIG. 5 illustrates that heat exchange between a person and the environment is carried out as a result of convection (Q_{conv}), radiation (Q_{rad}) and in the process of breathing (Q_{breath}) according to an embodiment of the disclosure. Biological tissue can be represented as a sequence of flat layers with a thickness of d, which have different thermal parameters (for example, density (r), heat capacity (C), thermal conductivity (k), etc.) that affect the transfer of heat from blood vessels to the skin surface. Resistance to heat transfer is exerted by all layers of biological tissue, but the fatty layer has the greatest resistance. The composition of the body of each person is unique, each person has individual metabolic characteristics, and all this affects how heat transfer will take place directly in the area where the electronic device is located.

The thermal properties of the skin and adjacent fat tissues correlate with overall body composition and mass index. Knowing these parameters, it is possible to evaluate how heat transfer occurs in the human body. In the disclosure, bioimpedancemetry is used to assess the composition of the human body. Measurement of morphology parameters is carried out by means of bioimpedance analysis. The thermal and mechanical properties of the skin and underlying tissues can be indirectly assessed using data taken from the bioimpedance sensor and profile data of the user. The profile data may include sex, age, height, and weight.

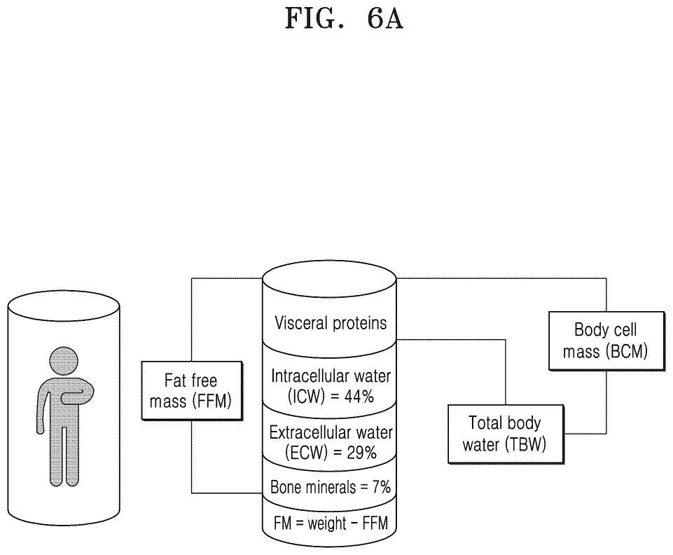

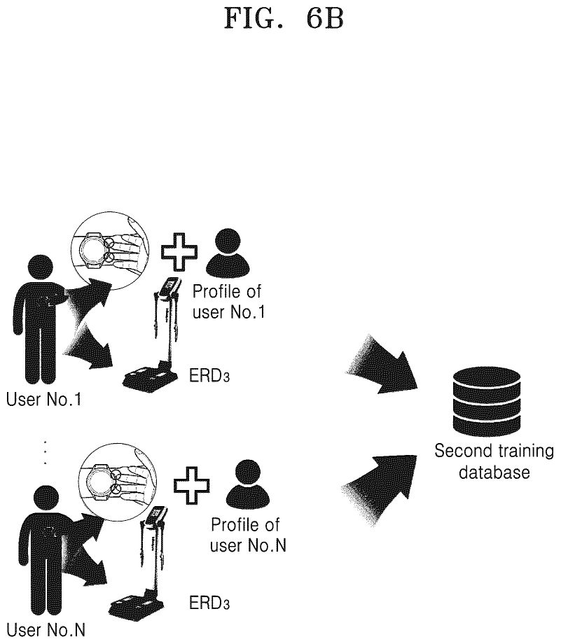

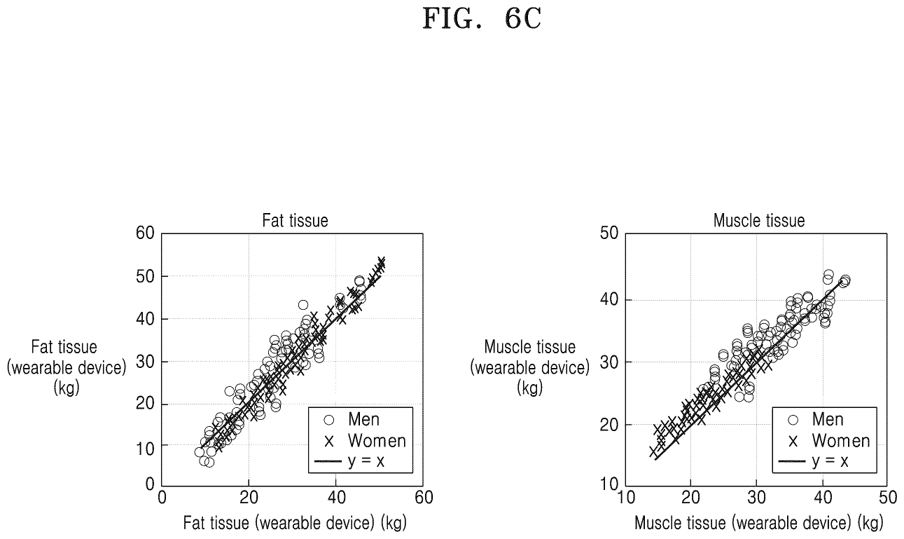

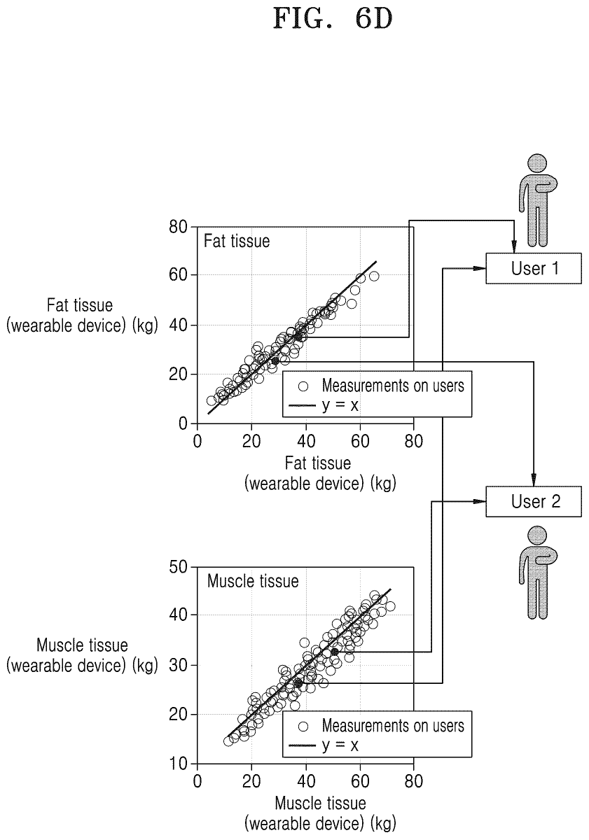

FIG. 6A illustrates the human body as a conductive cylinder and the corresponding scheme for representing a human body composition in bioimpedance analysis according to an embodiment of the disclosure.

Referring to FIG. 6A, the human body is represented as a conducting cylinder, and the corresponding scheme for representing the composition of the human body in bioimpedancemetry is also shown.

To take into account such factors, the disclosure provides for prediction of body morphology parameters of the user based on profile data of the which are user pre-entered into the electronic device and said at least one body impedance parameter of the user which is obtained by at least one bioimpedance sensor 207.

In the disclosure, this is achieved by means of a second prediction sub-algorithm that was created in advance as a result of neural network machine learning.

To train the second sub-algorithm for predicting the ability to determine the body morphology parameters of the user, a second training data base was formed. To do this, a second plurality of other users (test subjects) were recruited, who wore an electronic device (its test sample) on their wrist, having said at least one bioimpedance sensor 207. Each of the second plurality of other users entered a profile data of the user into the electronic device. Next, each of said second plurality of other users was subjected to measurement of at least one impedance parameter via said at least one bioimpedance sensor 207. The at least one impedance parameter may include at least one of a magnitude of body impedance of the user, a phase angle of body impedance of the user, and a magnitude of a contact impedance. The obtained at least one impedance parameter was used to determine the morphology parameters of each of said second plurality of other users: in particular, the magnitude of impedance was used to determine an amount of a fat tissue, an amount of a muscle tissue, an amount of water in the body, a phase angle of the impedance was used to determine a ratio of extracellular and intracellular water, a magnitude of the contact impedance was used to determine a condition of the skin. The condition of the user may include at least one of thickness of the user skin or wetness of the user skin

FIG. 6B illustrates the use of a third external reference device (ERD₃) in determining the body morphology parameters of the user according to an embodiment of the disclosure.

Referring to FIG. 6B, a third external reference device (ERD₃), such as the InBody body composition analyzer used in both medicine and fitness centers) was additionally used, which, for each of said second plurality of other users, made it possible to accurately determine a reference body composition. By means of a third external reference device, reference morphology parameters were determined for each of said second plurality of other users: a reference magnitude of impedance, which was used to determine an amount of a fat tissue, an amount of a muscle tissue, an amount of water in the body, a reference phase angle of impedance, which was used to determine a ratio of extracellular and intracellular water, a reference magnitude of a contact impedance module, which was used to determine a condition of the skin (dry or wet).

The morphology parameters determined for each of said second plurality of other users based on the readings of said at least one bioimpedance sensor 207 and the reference morphology parameters determined for each of said second plurality of other users based on the readings of the third reference device were also allocated in the second training database. Thus, the second training data base was formed, which was used to train the second prediction sub-algorithm.

The trained second sub-algorithm made it possible to predict the morphology parameters of the user based on the readings of said at least one bioimpedance sensor 207. The morphology parameters of the user may include an amount of fat tissue, a muscle tissue, water in the user body, a ratio of extracellular and intracellular water in the user body, and a state of the user skin.

The solution of the problem of predicting the body morphology parameters of the user was based on revealing empirical patterns in the training data allocated in the second database, using a machine method.

FIG. 6B illustrates the results of predicting morphology parameters (in particular, k_{fat}, k_{muscle}) for a second plurality of other users. In this study, a large number of test subjects were involved, among which were women and men, who had different body composition, different amount of fat and muscle tissue, different age, weight. The left graph shows the results obtained for a fat tissue and the right graph shows the results obtained for a muscle tissue. The x-axis represents the data calculated from the readings of the third external reference device (ERD₃) in kilograms, the y-axis shows the data calculated from the readings of the at least one bioimpedance sensor 207, in kilograms. The x=y line shows the correlation.

FIG. 6C illustrates the results of predicting morphology parameters using at least one bioimpedance sensor of an electronic device and using a third external reference device according to an embodiment of the disclosure.

Data shown in FIG. 6C corresponds to the case when the measurement of at least one impedance parameter by said at least one bioimpedance sensor 207 was performed for the upper part of the body (from left hand to right hand), while the measurement of impedance parameters was performed by the third external reference device (ERD₃) through the whole body. Other devices with a body composition function, such as a smart scale, can also be used as the third external device. Depending on the number of sensors used, the impedance measurements in such devices may only be through the legs and lower part, or through the entire body. The use of reference devices that provide measurement of impedance parameters through the whole body is the most preferable.

FIG. 6D illustrates the results of predicting body morphology parameters (composition) of a of the tested subject based on a clinical database containing information on 578 subjects according to an embodiment of the disclosure. The graph above shows the results of determining the amount of a fat tissue, the graph below shows the results of determining the amount of a muscle tissue, the x-axis plots the data calculated from the readings of the third external reference device, in kilograms, the y-axis plots the data calculated from the readings of said at least one bioimpedance sensor 207, in kilograms. The x=y line shows the correlation. Also FIG. 6D, 2 points are highlighted corresponding to two test subjects with the total mass, but different amounts of fat and muscle tissue: User 1 is a man with 35 kg of fat tissue and 28 kg of muscle tissue, and User 2 is a woman with 28 kg of fat tissue and 35 kg of muscle tissue. It can be seen that User 1 has a much higher ratio of fat tissue to muscle tissue (thicker subcutaneous fat) than User 2. Therefore, the transfer of bioheat from the blood vessels to the skin surface of User 1 will be hampered by a thicker layer of subcutaneous fat compared to User 2. It is known that the temperature of the skin surface varies by approximately 0.5° C., depending on what kind of body fat a person has, therefore, taking into account the morphology parameters makes it possible to increase the accuracy of determining the body core temperature of the user from the temperature of the skin surface.

The data presented in FIGS. 6C and 6D is obtained using a “ridge regression” machine learning model and a cross-validation performance evaluation method.

### 3) Determination of Peripheral Microcirculation Parameters of the User

As it is shown above, bioheat transfer also depends on the diameter of blood vessels, blood volume and blood flow velocity, which in turn can change under the influence of processes inside the body or external conditions. For example, due to cold, blood vessels can constrict, due to heat they can expand, affecting the speed of blood flow, the volume of blood flow, which will affect the transfer of heat. Therefore, the peripheral microcirculation parameters must also be taken into account when recalculating the body core temperature based on the temperature of the skin surface.

To define the body peripheral microcirculation of the user, the disclosure uses such a parameter as a body perfusion index, which defines blood filling, as well as features of the PPG signal shape, which defines the state of the human cardiovascular system.

In order to take into account such factors, the disclosure provides for prediction of peripheral microcirculation parameters based on the body perfusion parameters of the user, calculated from said at least two PPG signals with different wavelengths, obtained by at least one PPG sensor 208.

In the disclosure, said prediction is provided by a third prediction sub-algorithm that has been generated in advance as a result of neural network machine learning.

To train the third sub-algorithm for predicting the ability to determine the peripheral microcirculation parameters of the user, the third training data base was formed. For each of said third plurality of other users wearing the electronic device (its test piece) on their wrist, at least two PPG signals at different wavelengths were obtained by said at least one PPG sensor 208.

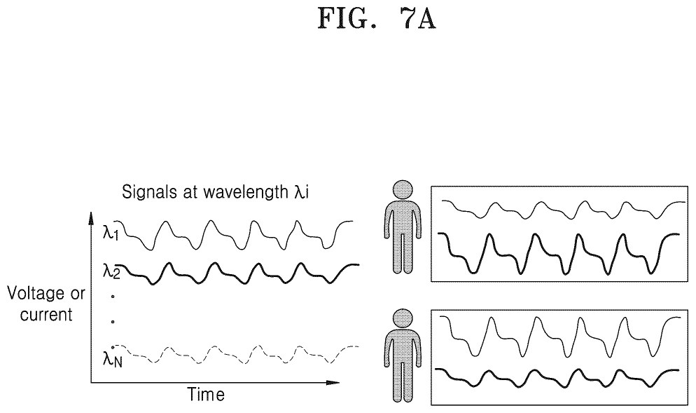

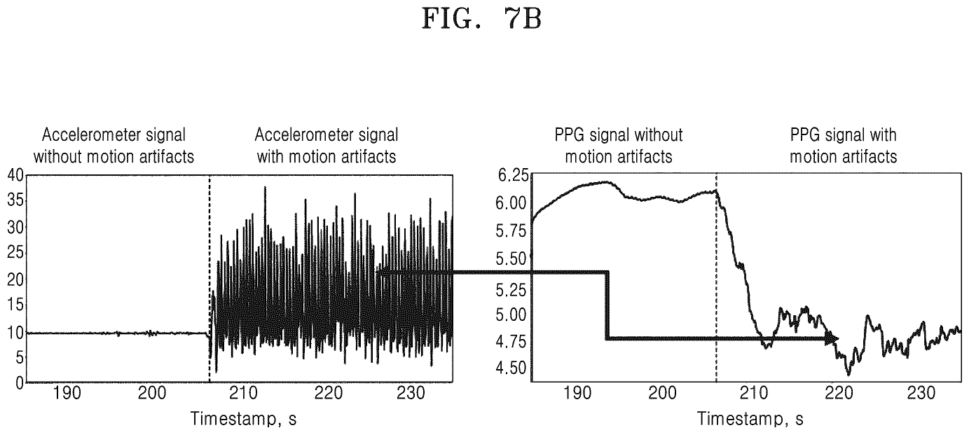

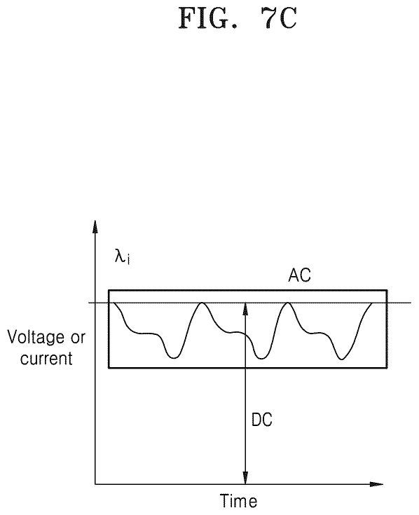

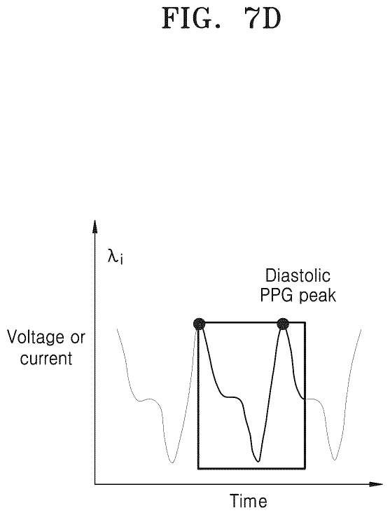

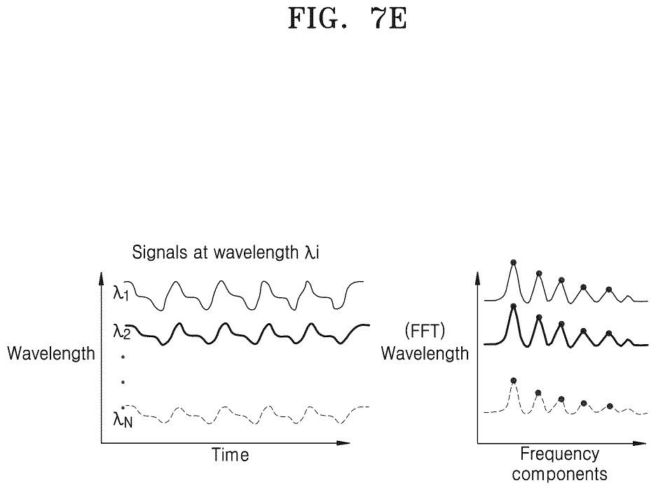

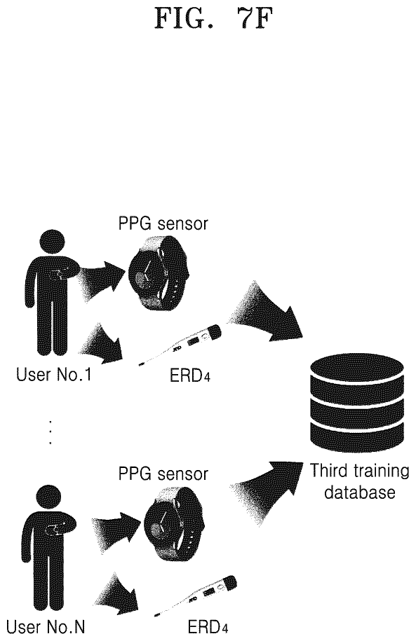

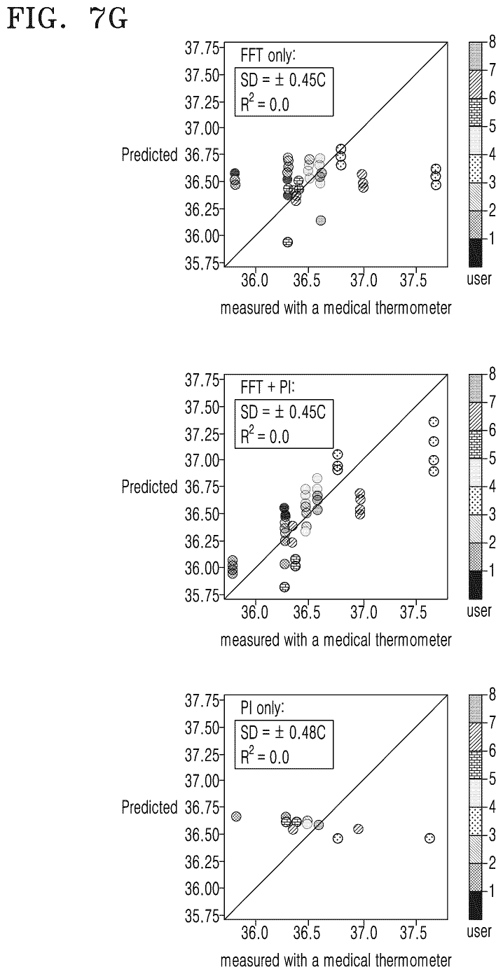

FIG. 7A illustrates the reasons for using PPG signals with different wavelengths when predicting peripheral microcirculation parameters according to an embodiment of the disclosure.

It is shown with reference to FIG. 7A that in the disclosure the measurement of said at least two PPG signals with different wavelengths (I₁, I₂, . . . I_{N}) makes it possible to take into account a skin tone of a person, i.e. how light or darker, tanned their skin is, and, depending on the skin tone, detect a higher quality PPG signal. The selection of a higher quality PPG signal is based on a test of the signal-to-noise ratio. In general, a high melanin content in darker skin tones reduces the signal-to-noise ratio for the green wavelength, a low melanin content in lighter skin tones increases the signal-to-noise ratio for the green wavelength. Thus, for fair skin, it is preferable to use the PPG signal at the green wavelength, as it has a better signal-to-noise ratio, and for a person with dark skin, it is preferable to use other wavelengths, for example, infrared, red wavelength, to ensure good PPG signal quality.

FIG. 7B illustrates filtration of said at least two PPG signals from motion artifacts (finger movements, trembling, etc.) according to the accelerometer data in order to get rid of motion artifacts and work with a higher quality filtered signal according to an embodiment of the disclosure.

FIG. 7C illustrates the separation of PPG signals into time-constant and non-time-constant components according to an embodiment of the disclosure.

It is shown with reference to FIG. 7C that said at least two PPG signals with different wavelengths are separated into time-constant and non-time-constant components (alternating current (AC) and direct current (DC) components) of the PPG time series, in addition, the AC and DC components of the PPG time series are separated into separate time segments (PPG pulses) according to the position of diastolic AC peaks (see FIG. 7D).

FIG. 7D illustrates the separation of the PPG signal into separate time segments (PPG pulses) according to an embodiment of the disclosure.

FIG. 7E illustrates the transformation of the time-constant and non-time-constant components of the PPG time series into a frequency domain according to an embodiment of the disclosure.

It is shown with reference to FIG. 7E that said time-constant and non-time-constant components of the PPG time series are transformed into the frequency domain with subsequent extraction of the amplitude-phase harmonic characteristics of the signals. For example, a Fourier transform can be performed where each PPG pulse is transformed into a set of Fourier amplitudes and phases and thus a set of 10 numbers [A1:A5, P1:P5] can contain all functions (information) for the machine learning model used to predict the peripheral microcirculation parameters of the user.

PPG signal waveform feature extraction is based on computing the first five Fast Fourier Transform (FFT) coefficients (amplitudes A and phases P)—PPG waveform features and perfusion indices (PI) for each wavelength, it can be performed as shown in table 2.

TABLE 2

| | PI | A1 | P1 | A2 | P2 | . . . |
|---|---|---|---|---|---|---|
| λ₁ | $\frac{{AC}_{1}}{{DC}_{1}}$ | amplitude value of | phase value of the | amplitude value of | phase value of the 2nd | |
| | | the 1st FFT | 1st FFT | the 2nd FFT | FFT harmonic | |
| | | harmonic | harmonic | harmonic | | |
| λ₂ | $\frac{{AC}_{2}}{{DC}_{2}}$ | amplitude value of | phase value of the | amplitude value of | phase value of the 2nd | |
| | | the 1st FFT | 1st FFT | the 2nd FFT | FFT harmonic | |
| | | harmonic | harmonic | harmonic | | |
| . . . | | | | | | |

Thus, the peripheral microcirculation is a function of f (PI=AC/DC; FFT coefficients of the PPG waveform).The disclosure is not limited to application of the Fourier transform, but other transforms such as the Hilbert-Huang transform and the Wavelet transform may be used.

At the first stage of the Hilbert-Huang transform, the PPG signal is decomposed into a number of components, which are called empirical modes (EM), at the second stage, the Hilbert transform is applied to the resulting decomposition. The main informative features of the Hilbert-Huang transform are the amplitude, instantaneous frequency and phase of the empirical modes obtained as a result of the empirical mode decomposition of the original signal.

In the Wavelet transform, an integral transform is performed, which is a convolution of the wavelet function with the PPG signal. The Wavelet transform transforms the PPG signal from time to frequency-time representation. Wavelet transformation algorithms allow taking into account local changes in PPG signals and carrying information in a three-dimensional format—amplitude, frequency, time.

For each of said third plurality of other users, the obtained PPG signals, the results of their intermediate processing, as well as the data presented in Table 2 were allocated in the third training database.

FIG. 7F illustrates the use of the fourth external reference device (ERD₄) in predicting the body morphology parameters of the user according to an embodiment of the disclosure.

Additionally, a fourth external reference device (ERD₄) (FIG. 7F) was used, for example, a medical thermometer, which for each of said third plurality of other users allowed accurate measurement of body temperature, for example, an electronic thermometer, which obtained the temperature in the mouth or in the armpit. The data from the fourth external reference device was also allocated in the third training database. Thus, the third training data base was formed, which was used to train the third prediction sub-algorithm.

The trained third prediction sub-algorithm made it possible to predict the peripheral microcirculation parameters based on the readings of the at least one PPG sensor 208 with different wavelengths.

The solution of the problem of predicting the peripheral microcirculation was based on revealing empirical patterns in the training data allocated in the third database, by machine.

FIG. 7G illustrates the effect of a combination of perfusion scores and PPG waveform features on the accuracy of body core temperature prediction according to an embodiment of the disclosure.

The results shown in FIG. 7G demonstrate the effect of a combination of perfusion scores and PPG waveform features on the accuracy of body core temperature prediction. The graphs show data from 8 subjects who used an electronic device, specifically a smartwatch. On the graphs, the x-axis plots the temperatures obtained by a medical thermometer, ° C., the y-axis plots the temperatures calculated by the third prediction sub-algorithm, ° C.

On the left of the graph, only features associated with the PPG waveform detected by FFT are used for prediction, on the right of the graph, only perfusion parameters (PI) are used for prediction, in the middle graph, features associated with the waveform of the PPG signal detected by FFT are used, and perfusion parameters (PI). It can be seen that when the features are used together (the graph in the center), the accuracy of determining the body core temperature increases.

The data presented in FIG. 7G are obtained using the “ridge regression” machine learning model and the method of evaluating the effectiveness of cross-validation.

### 4) Predicting the Body Core Temperature of the User

The first, second, and third training databases that were used for machine learning of said prediction sub-algorithms were combined into a common training database. In addition, additional reference body temperatures obtained with external reference measuring instruments (ear, mouth and forehead medical thermometers) have been added to this common training database. As a result of the studies, a unique clinical database was collected containing information on a variety of test subjects who had a different body composition, age, gender, a different peripheral microcirculation, among them were healthy people and people with a temperature of >37,2° C., etc.

This common database was used to train the fourth prediction sub-algorithm to be able to predict the body core temperature of the test subject by predicting based on at least the local microclimate parameters around the electronic device, the body morphology parameters (composition) of the user, the peripheral microcirculation parameters of the user determined in steps 1)-3) as detailed above.

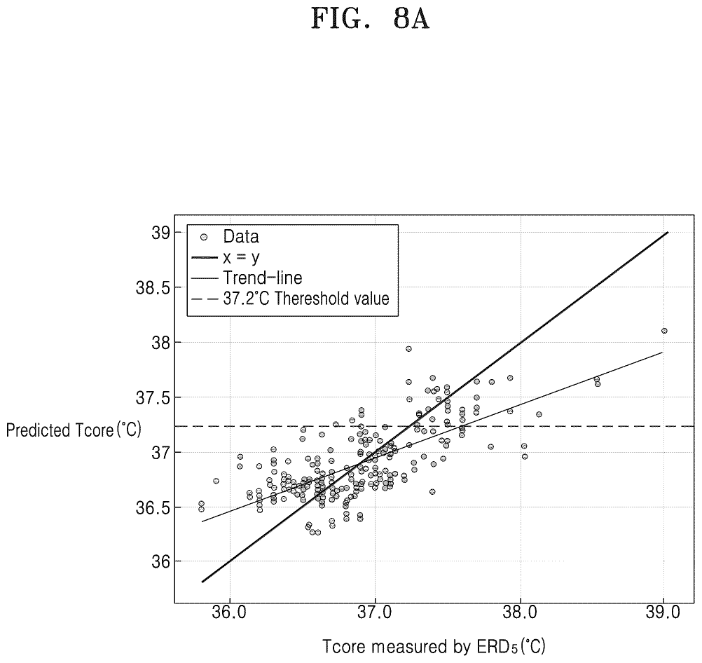

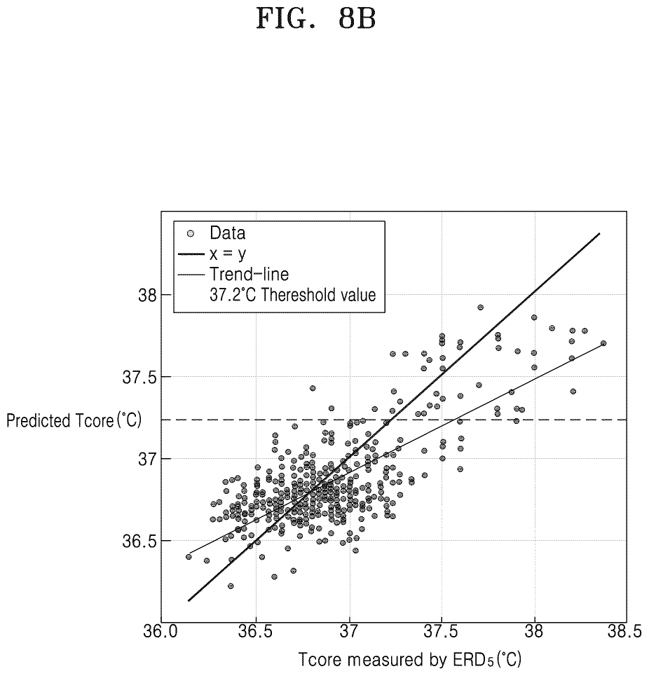

FIG. 8A illustrates the results of predicting the body core temperature of the user from the clinical database containing information on 271 tests performed on 88 test subjects (62 of them were febrile) according to an embodiment of the disclosure. When collecting reference data, external reference devices (ERD₅) were used—medical thermometers, by means of which the reference body core temperature of the test subject was determined (in the ears, armpit, on the forehead), different environmental conditions (humidity and temperature) took place.

In the graph, the x-axis shows the body core temperature of the test subject obtained by a medical thermometer (ERD), the y-axis shows the body core temperature predicted by the prediction algorithm. The x=y line shows the correlation. A trend line is an empirical pattern identified by a machine. In this case, a “ridge regression” machine learning model and a performance evaluation method—cross-validation (exclusion of one user) were used. The accuracy characteristics of the machine learning model are shown in Table 3.

TABLE 3
Machine Learning Model Accuracy Characteristics

| | |
|---|---|
| MAE, ° C. | 0.267 |
| RMSE, ° C. | 0.342 |
| R2corr | 0.497 |
| Bias, ° C. | 0.000005 |
| Error SD, ° C. | 0.342 |

The data presented in Table 3 clearly demonstrates that in this case, the body core temperature of the test subject was predicted based on at least a measurement data set taken from the sensors of the electronic device, with an accuracy of ±0.267° C. (MAE=0.267° C.) relative to readings of a medical thermometer.

FIG. 8B illustrates the results of predicting the body core temperature of a test subject from a clinical database containing information on 484 tests performed on 266 test subjects according to an embodiment of the disclosure. When collecting reference data, external reference devices (ERD), medical thermometers, were used by means of which the reference body core temperature of the test subject was determined (in the ears, armpit, on the forehead), different environmental conditions (humidity and temperature) took place.

In the graph, the x-axis shows the body core temperature of the test subject obtained by a medical thermometer (ERD), the y-axis shows the body core temperature predicted by the prediction algorithm. The x=y line shows the correlation. A trend line is an empirical pattern identified by a machine. In this case, a “ridge regression” machine learning model and a performance evaluation method—cross-validation (exclusion of one user) were used. The accuracy characteristics of the machine learning model are shown in Table 4.

TABLE 4
Machine Learning Model Accuracy Characteristics

| | |
|---|---|
| MAE, ° C. | 0.2 |
| RMSE, ° C. | 0.252 |
| R2corr | 0.554 |
| Bias, ° C. | −0.038 |
| Error SD, ° C. | 0.249 |

The data presented in Table 4 clearly demonstrates that the body core temperature of a test subject can be predicted based on at least a measurement data set taken from the sensors of an electronic device with an accuracy of ±0.2° C. (MAE=0.2° C.) relative to the indications of a medical thermometer. Based on the information presented in FIGS. 8A and 8B, it can be seen that as information is accumulated in the clinical database with the results of measurements on the tested subjects, the prediction accuracy increases. In particular, the electronic device can provide the measurement of body core temperature of the user with an accuracy of, for example, ±0.3° C. or less from the value of the user body core temperature obtained by a medical thermometer, in particular, with an accuracy of ±0.2° C. or less of values of the body core temperature of the user, measured by a medical thermometer. Thus, the electronic device is able to determine the body core temperature of the user with an accuracy comparable to the accuracy of temperature determination using medical thermometers.

The solution to the problem of predicting the body core temperature of the user was based on revealing empirical patterns in the training data allocated in a common database using a machine method.

Thus, the created prediction algorithm comprises first, second, third and fourth prediction sub-algorithms trained as detailed above. In the embodiment of the disclosure described above, the prediction algorithm is created for the electronic device located on the wrist, because training of the prediction algorithm was based on data captured by the sensors of the electronic device located on the wrist. In some embodiments of the disclosure, it is possible to train a prediction algorithm in view of location of the electronic device on other parts of the body, such as a finger. The prediction algorithm described in this document may be implemented in software including one or more instructions that may be executed by the at least one processor 203 of the electronic device 201.

In the prediction algorithm training process described above, profile data of other users (test subjects), measurement data sets pre-measured from other users, and reference data sets pre-measured by external reference devices ERD₁ to ERD₅ were used as detailed above.

In general, in the process of machine learning of the prediction algorithm, the following methods of selection and processing of features and corresponding discrete frequencies can be used:
The Relief-F method calculates and normalizes the vector of feature weights, and then selects the features the weight of which exceeds the value of said threshold.
Correlation-based Feature Selection (CFS) combines a scoring formula with an appropriate correlation score and a heuristic search strategy.
The Fast Correlation Based Filter method starts working with a full set of features, uses a measure of symmetric uncertainty to determine the dependencies between features and allows selecting a subset by searching and sequentially excluding less informative features.
The Sequential forward feature selection (SFFS) method, at each iteration, adds a feature to the set that provides the best recognition efficiency for this iteration. The Mutual Information method determines a non-linear correlation dependence instead of calculating the “feature-feature” and “feature-label” Pearson correlation.
Basic algorithms for machine learning (artificial intelligence). As a working solution, a combination of these methods and any derivative methods based on the basic algorithm can be used:
Decision Trees/Random Forests;
Support Vector Machines;
Naive Bayes method;
Linear Regression;
Logistic Regression;
Deep learning methods—artificial neural networks.

Said “ridge regression” machine learning algorithm mentioned with reference to FIGS. 4F, 6C, 6D, 7G, and 8 belongs to the “linear regression” class, while cross-validation is a standard step in any of the machine learning algorithms, when the value of the algorithm error is checked on data that did not participate in building the model itself.

Due to the fact that when determining the body core temperature of the user in the disclosure, at least the local microclimate parameters of the electronic device-user body part system, the body morphology parameters (composition) of the user and the peripheral microcirculation parameters of the user are taken into account, the accuracy of determining the body core temperature of the user using an electronic device is ±0.2° C. relative to the body core temperature of the user, obtained using medical thermometers.

In an embodiment of the disclosure, it is possible to train a prediction algorithm taking into account the additional data.

For example, when training the prediction algorithm, data on humidity, weather conditions, time of day, etc. can be additionally taken into account. In this case, humidity data can be acquired from a humidity sensor that may be part of the electronic device 201, or data on humidity, weather conditions, time of day can be acquired from the Internet. By taking into account additional environmental parameters, it is possible to further improve the accuracy of determining the body core temperature of the user.

In an embodiment of the disclosure, prediction can be improved by more accurate measurement of peripheral microcirculation parameters. This can be achieved by providing the at least one PPG sensor 208 with an additional radiation source 221 at a wavelength of, for example, 850 nm, which is preferred for measuring perfusion parameters. In a preferred embodiment of the disclosure, this additional radiation source 221 may have a power of, for example, 1 mW.

In an embodiment of the disclosure, the prediction can be improved by additionally taking into account previous measurements made directly to the user of the electronic device and accumulated, for example, in the memory of the electronic device. For example, when the electronic device determines the body core temperature of the user, along with data taken from the sensors 205, 206, 207, 208 of the device, data on previously predicted morphology parameters and peripheral microcirculation parameters for this user can also be taken into account.

In an embodiment of the disclosure, the prediction algorithm may operate without profile data of the user, i.e. using only data from the sensors of the electronic device. Obviously, in this case, a lower accuracy in determining the body core temperature of the user will be achieved. Immediately after filling in the profile data of the user, the algorithm begins to take it into account, respectively, the accuracy of determining the body core temperature of the user increases. If necessary, the user can change the profile data (e.g., weight, age) of the user, since the prediction algorithm is designed to track and take into account these changes.

The electronic device can operate in the following modes: measurement mode, which performs a single determination of the body core temperature of the user; a continuous monitoring mode in which the determination of the body core temperature of the user is performed with some periodicity for some predetermined period of time. The latter mode may be relevant for a person with a feverish condition, or an athlete, since for some sports a decrease or increase in temperature during exercise is typical.

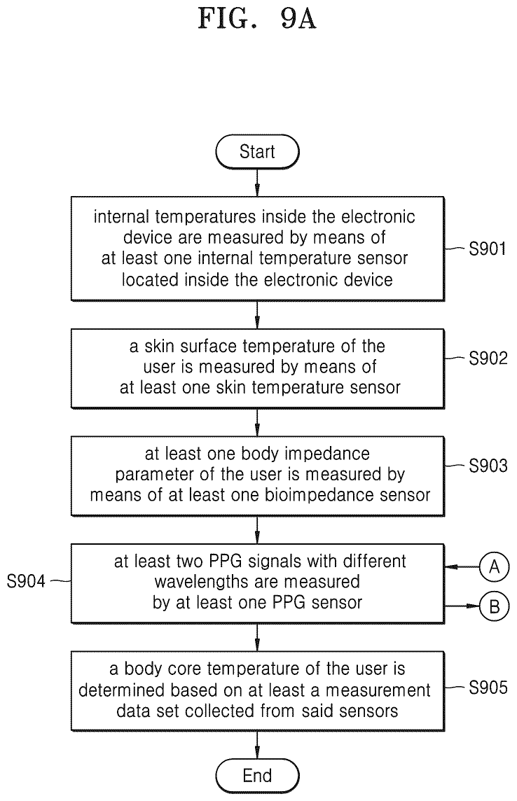

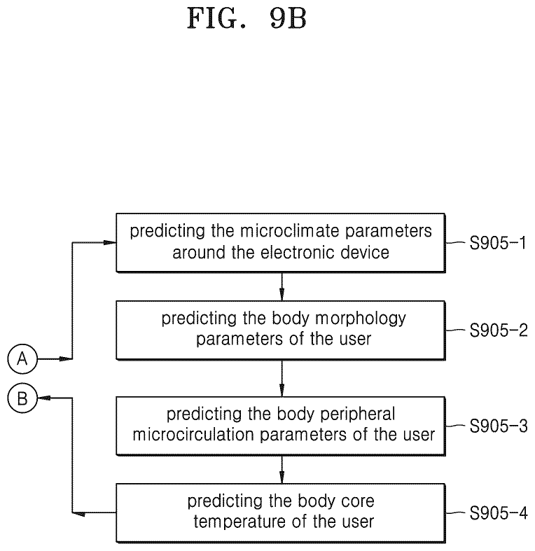

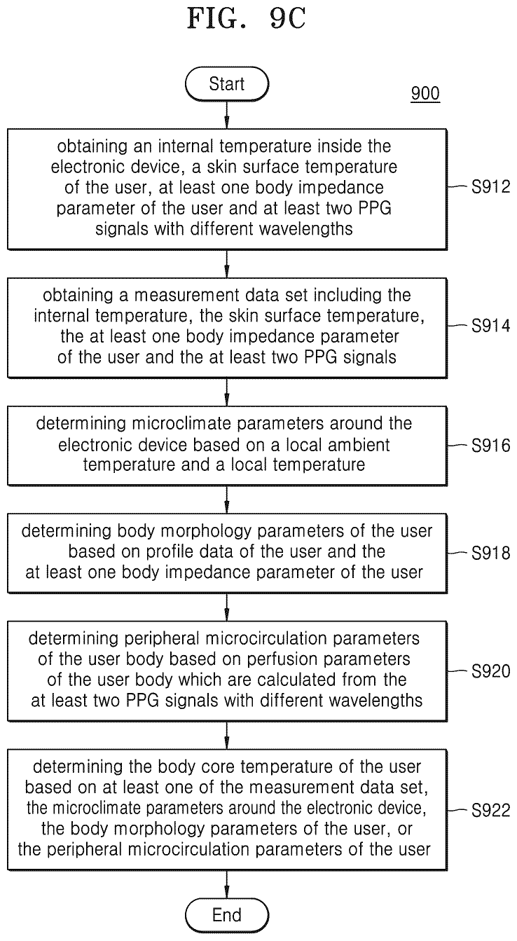

Next, with reference to FIGS. 2A to 2E, 9A, and 9C, steps for carrying out the operation method of the electronic device 201 of the disclosure will be described.

FIG. 9A illustrates schematically the sequence of steps in the method of operation of an electronic device with a function of determining a body core temperature of the user according to an embodiment of the disclosure.

Referring to FIG. 9A, at the first step of the method S901, internal temperatures inside the electronic device, e.g. T_{hous.}, T_{proc.}, T_{bat.}, are measured by means of at least one internal temperature sensor 205 located inside the electronic device 201.

At the second step of the method S902, a skin surface temperature of the user, T_{skin surf.}, is measured by means of at least one skin temperature sensor 206,

At the third step of the method S903, at least one body impedance parameter of the user is measured by means of at least one bioimpedance sensor 207,

At the fourth step of the method S904 at least two PPG signals with different wavelengths are measured by at least one PPG sensor 208.

At the fifth step of the method S905, a body core temperature, T_{core}, of the user is determined based on at least a measurement data set collected from said sensors 205, 206, 207, 208.

FIG. 9B illustrates the sub-steps of the prediction step performed in the method of operation of the electronic device with a function of determining the body core temperature of the user according to an embodiment of the disclosure.

Referring to FIG. 9B, in an embodiment of the disclosure, the fifth method step S905 may comprise the following sub-steps:
predicting the microclimate parameters around the electronic device 201, comprising predicting a local ambient temperature, T_{loc.amb.}, around the wearable electronic device 201 based on the internal temperatures inside the electronic device, for example, T_{hous.}, T_{proc.}, T_{bat.}, measured by said at least one internal temperature sensor 205, and then predicting the local temperature between the electronic device and the user skin, T_{lop.device}, based on said local ambient temperature, T_{loc.amb.}, and the skin surface temperature of the user, T_{skin surf.}, measured by said at least one skin temperature sensor 206 at sub-step S905-1;
predicting the body morphology parameters (e.g., k_{fat}, k_{muscle}) of the user based on the profile data of the user which are pre-entered into the electronic device 201 and said at least one body impedance parameter of the user which is measured by said at least one bioimpedance sensor 207 at sub-step S905-2;
predicting the body peripheral microcirculation parameters of the user based on the body perfusion parameters of the user which are calculated from said at least two PPG signals with different wavelengths measured by said at least one PPG sensor 208 at sub-step S905-3; and
predicting the body core temperature of the user based on at least said microclimate parameters around the electronic device, said body morphology parameters of the user and said body peripheral microcirculation parameters of the user at sub-step S905-4.

In an embodiment of the disclosure, when calculating perfusion parameters in sub-step S905-3 of the method, said at least two PPG signals with different wavelengths are filtered from motion artifacts, said at least two PPG signals with different wavelengths are separated into time-constant and non-time-constant components of the time series and transform these time series into a frequency domain or a frequency-time domain with subsequent extraction of its amplitude-phase harmonic characteristics. Wherein, said time series are transformed into the frequency domain by means of the Fourier transform or the Hilbert-Huang transform, or are transformed into the frequency-time domain by the Wavelet transform.

In an embodiment of the disclosure, when predicting in sub-steps S905-1 to S905-4 of the method, profile data of other users, measurement data sets pre-measured from other users, and reference data sets pre-measured by external reference measurement means are further taken into account.

In the method, prediction is based on applying a prediction algorithm trained as detailed above.

FIG. 9C illustrates an example method 900 for determining a body core temperature of the user according to an embodiment of the disclosure. According to an embodiment of the disclosure, a method 900 for determining a body core temperature of a user is provided. The method 900 may include obtaining an internal temperature (e.g. T_{hous.}, T_{proc.}, T_{bat.}) inside the electronic device at step S912, a skin surface temperature of the user (e.g. T_{skin surf.}), at least one body impedance parameter of the user and at least two PPG signals with different wavelengths. The method 900 may include obtaining a measurement data set including the internal temperature, the skin surface temperature at step S914, the at least one body impedance parameter of the user and the at least two PPG signals. The method 900 may include determining microclimate parameters around the electronic device based on a local ambient temperature (e.g. T_{loc.amb}) and a local temperature at step S916. The method 900 may include determining body morphology parameters of the user (e.g., k_{fat}, k_{muscle}) based on profile data of the user and the at least one body impedance parameter of the user at step S918. The method 900 may include determining peripheral microcirculation parameters of the user body based on perfusion parameters of the user body which are calculated from the at least two PPG signals with different wavelengths at step S920. The method 900 may include determining the body core temperature of the user (e.g. T_{core}) based on at least one of the measurement data set at step S922, the microclimate parameters around the electronic device, the body morphology parameters of the user, or the peripheral microcirculation parameters of the user.

According to an embodiment of the disclosure, the determining the microclimate parameters may include determining the microclimate parameters based on at least one sub-algorithm trained by using profile data of other users, measurement data sets pre-measured from other users, and reference data sets pre-measured by external reference measurement means. According to an embodiment of the disclosure, the determining the body morphology parameters may include determining the body morphology parameters based on the at least one sub-algorithm trained by using profile data of other users, measurement data sets pre-measured from other users, and reference data sets pre-measured by external reference measurement means. According to an embodiment of the disclosure, the determining the peripheral microcirculation parameters may include determining the peripheral microcirculation parameters based on the at least one sub-algorithm trained by using profile data of other users, measurement data sets pre-measured from other users, and reference data sets pre-measured by external reference measurement means. According to an embodiment of the disclosure, the determining the body core temperature may include determining the body core temperature, based on the at least one sub-algorithm trained by using profile data of other users, measurement data sets pre-measured from other users, and reference data sets pre-measured by external reference measurement means.

According to an embodiment of the disclosure, the at least one body impedance parameter of the user may include a magnitude of body impedance of the user, a phase angle of body impedance of the user, a magnitude of a contact impedance.

According to an embodiment of the disclosure, the determining the body core temperature of the user may include determining, based on the body morphology parameters of the user, an amount of a fat tissue, a muscle tissue, water in the user body, a ratio of extracellular and intracellular water in the user body and a condition of the user skin.

According to an embodiment of the disclosure, the determining peripheral microcirculation parameters of the user body may include filtering the at least two PPG signals with different wavelengths from motion artifacts, separating the at least two PPG signals with different wavelengths into time-constant and non-time-constant components of time series of the at least two PPG signals and transforming the time series into a frequency domain or a frequency-time domain based on subsequent extraction of an amplitude-phase harmonic characteristics of the signals.

In an embodiment of the disclosure, the at least one PPG sensor comprises at least one radiation source and at least one radiation receiver.

In an embodiment of the disclosure, the at least one radiation source comprises a light emitting diode (LED).

In an embodiment of the disclosure, the electronic device further comprises a housing that houses a processor and a battery.

In an embodiment of the disclosure, the at least one internal temperature sensor is configured for measuring a housing temperature, a processor temperature, and a battery temperature.

In an embodiment of the disclosure, the at least one skin temperature sensor is an IR temperature sensor or a resistance temperature sensor.

In an embodiment of the disclosure, the electronic device further comprises an input device configured to input a profile data of the user, the profile data of the user comprising a gender, an age, a height, and a weight of the user, and an output device configured to output information about the body core temperature of the user.

In an embodiment of the disclosure, the electronic device is designed to be located on the wrist.

In an embodiment of the disclosure, the electronic device is a smart device.

In an embodiment of the disclosure, the electronic device is a smart watch or a fitness bracelet.

In an embodiment of the disclosure, the electronic device further comprises memory configured to store the profile data of the user, measurement data sets, dates and times of collection of each measurement data set, and values of the body core temperatures of the user which are determined by the device for each measurement data set.

In an aspect of the disclosure, a method for operating the electronic device is provided, comprising: measuring temperatures within the electronic device by means of at least one internal temperature sensor disposed within the electronic device; measuring a skin surface temperature of the user by at least one skin temperature sensor; measuring at least one body impedance parameter of the user by means of at least one bioimpedance sensor; measuring at least two PPG signals with different wavelengths by means of at least one PPG sensor, and determining a body core temperature of the user based on at least a measurement data set collected from the sensors.

In an embodiment of the disclosure, the measurement data set includes temperatures inside the electronic device, the skin surface temperature of the user, the at least one body impedance parameter of the user, and the at least two PPG signals with different wavelengths.

In an embodiment of the disclosure, the method further comprises: predicting microclimate parameters around the electronic device, comprising predicting a local ambient temperature around the electronic device based on the temperatures inside the electronic device measured by the at least one internal temperature sensor, and then predicting a local temperature between by the electronic device and the user skin based on the local ambient temperature and the skin surface temperature of the user which is measured by the at least one skin temperature sensor; predicting the body morphology parameters of the user based on the profile data of the user which are pre-entered into the electronic device and the at least one body impedance parameter of the user which is measured by the at least one bioimpedance sensor; predicting the peripheral microcirculation parameters of the user body based on the body perfusion parameter of the user which are calculated from the at least two PPG signals with different wavelengths measured by the at least one PPG sensor; and predicting the body core temperature based on at least the microclimate parameters around the electronic device, the body morphology parameters of the user, and the body peripheral microcirculation parameters of the user.

In an embodiment of the disclosure, the when predicting, profile data of other users, measurement data sets pre-measured from other users, and reference data sets pre-measured by external reference measurement means are taken into account.

In an embodiment of the disclosure, the at least one body impedance parameter of the user includes a magnitude of the body impedance of the user, a phase angle of body impedance of the user, a magnitude of a contact impedance.

In an embodiment of the disclosure, the body morphology parameters of the user define an amount of a fat tissue, muscle tissue, water in the user body, a ratio of extracellular and intracellular water in the user body, a condition of the user skin.

In an embodiment of the disclosure, when calculating the perfusion parameters, the at least two PPG signals with different wavelengths are filtered from motion artifacts, the at least two PPG signals with different wavelengths are separated into time-constant and non-time-constant components of the time series, and these time series are transformed into the frequency domain or a frequency-time domain with subsequent extraction of their amplitude-phase harmonic characteristics.

In an embodiment of the disclosure, the time series are transformed to the frequency domain by a Fourier transform or a Hilbert-Huang transform, or transformed to time-frequency by a Wavelet transform.

In a third aspect of the disclosure, there is provided a system comprising the electronic device and a remote server and/or cloud storage, the system having at least one communication channel for transferring data between the electronic device and the remote server and/or cloud storage.

In an embodiment of the disclosure, the system is configured to control a function of determining a body core temperature of a user from other electronic devices via a remote server and/or cloud storage.

In an embodiment of the disclosure, the remote server and/or cloud storage are/is further configured to store profile data of the user, measurement data sets, dates and times of collection of each measurement data set, and body core temperatures of the user which are determined by the electronic device for each measurement data set, as a user database.

In an embodiment of the disclosure, the user database is configured to access user data from other electronic devices via a remote server and/or cloud storage.

In an embodiment of the disclosure, the system is further configured to use data collected in the user database to specify the predicting when determining the body core temperature of the user.

According to an embodiment of the disclosure, an electronic device for determining a body core temperature of a user is provided. The electronic device 201 may include at least one internal temperature sensor configured to measure an internal temperature inside the electronic device, at least one skin temperature sensor configured to measure a skin surface temperature of the user, at least one bioimpedance sensor configured to measure at least one body impedance parameter of the user, at least one photoplethysmogram (PPG) sensor configured to measure at least two photoplethysmogram (PPG) signals with different wavelengths, memory storing instructions and profile data of the user, and at least one processor. The at least one processor may be configured to execute the instructions to obtain the internal temperature inside the electronic device, the skin surface temperature of the user, the at least one body impedance parameter of the user and the at least two PPG signals with different wavelengths. The at least one processor may be configured to execute the instructions to obtain a measurement data set including the internal temperature, the skin surface temperature, the at least one body impedance parameter of the user and the at least two PPG signals. The at least one processor may be configured to execute the instructions to determine microclimate parameters around the electronic device based on a local ambient temperature and a local temperature. The at least one processor may be configured to execute the instructions to determine body morphology parameters of the user based on the profile data of the user and the at least one body impedance parameter of the user. The at least one processor may be configured to execute the instructions to determine peripheral microcirculation parameters of the user body based on perfusion parameters of the user body which are calculated from the at least two PPG signals with different wavelengths. The at least one processor may be configured to execute the instructions to determine the body core temperature of the user based on at least one of the measurement data set, the microclimate parameters around the electronic device, the body morphology parameters of the user, or the peripheral microcirculation parameters of the user

According to an embodiment of the disclosure, the at least one processor may be configured to execute the instructions to determine the local ambient temperature around the electronic device based on the internal temperature inside the electronic device. The at least one processor may be configured to execute the instructions to determine the local temperature between the electronic device and the user skin based on the local ambient temperature and the skin surface temperature of the user which is measured by the at least one skin temperature sensor.

According to an embodiment of the disclosure, the at least one processor may be configured to execute the instructions to determine the microclimate parameters based on at least one sub-algorithm trained by using profile data of other users, measurement data sets pre-measured from other users, and reference data sets pre-measured by external reference measurement means. The at least one processor may be configured to execute the instructions to determine the body morphology parameters based on the at least one sub-algorithm trained by using profile data of other users, measurement data sets pre-measured from other users, and reference data sets pre-measured by external reference measurement means. The at least one processor may be configured to execute the instructions to determine the peripheral microcirculation parameters based on the at least one sub-algorithm trained by using profile data of other users, measurement data sets pre-measured from other users, and reference data sets pre-measured by external reference measurement means. The at least one processor may be configured to execute the instructions to determine the body core temperature, based on the at least one sub-algorithm trained by using profile data of other users, measurement data sets pre-measured from other users, and reference data sets pre-measured by external reference measurement means.

According to an embodiment of the disclosure, the at least one body impedance parameter of the user includes a magnitude of body impedance of the user, a phase angle of body impedance of the user, a magnitude of a contact impedance.

According to an embodiment of the disclosure, the at least one processor may be configured to execute the instructions to determine, based on the body morphology parameters of the user, an amount of a fat tissue, a muscle tissue, water in the user body, a ratio of extracellular and intracellular water in the user body and a condition of the user skin.

According to an embodiment of the disclosure, the at least one processor may be configured to execute the instructions to filter the at least two PPG signals with different wavelengths from motion artifacts. The at least one processor may be configured to execute the instructions to separate the at least two PPG signals with different wavelengths into time-constant and non-time-constant components of time series of the at least two PPG signals. The at least one processor may be configured to execute the instructions to transform the time series into a frequency domain or a frequency-time domain based on subsequent extraction of an amplitude-phase harmonic characteristics of the signals.

According to an embodiment of the disclosure, the at least one processor may be configured to execute the instructions to transform the time series into the frequency domain or a frequency-time domain by using at least one of a Fourier transform, a Hilbert-Huang transform and a Wavelet transform.

According to an embodiment of the disclosure, A computer readable medium containing instructions which are executable by a processor to perform a method for determining a body core temperature of a user is provided. The method may include obtaining an internal temperature inside the electronic device, a skin surface temperature of the user at least one body impedance parameter of the user and at least two PPG signals with different wavelengths. The method may include obtaining a measurement data set including the internal temperature, the skin surface temperature, the at least one body impedance parameter of the user and the at least two PPG signals. The method may include determining microclimate parameters around the electronic device based on a local ambient temperature and a local temperature. The method may include determining body morphology parameters of the user based on profile data of the user and the at least one body impedance parameter of the user. The method may include determining peripheral microcirculation parameters of the user body based on perfusion parameters of the user body which are calculated from the at least two PPG signals with different wavelengths. The method may include determining the body core temperature of the user based on at least one of the measurement data set, the microclimate parameters around the electronic device, the body morphology parameters of the user, or the peripheral microcirculation parameters of the user.

It is an object of the disclosure to create electronic devices with a function of determining a body core temperature of a user based on data taken from sensors of the electronic devices.

The technical result of the disclosure is to increase an accuracy of determining a body core temperature of a user using an electronic device.

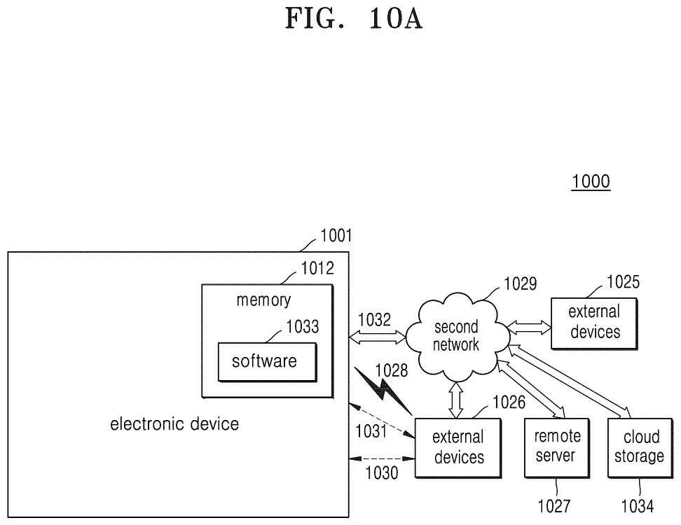

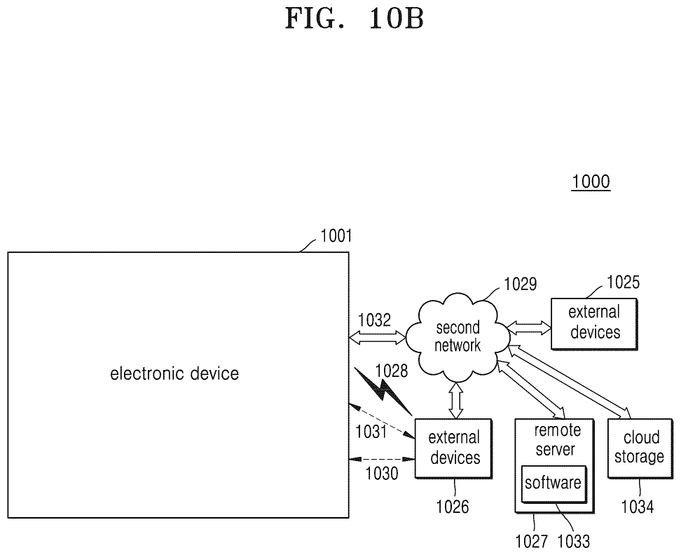

Next, with reference to FIGS. 2A to 2E, 10A, and 10B, a system 1000 will be described comprising the electronic device 201, 1001 and a remote server 1027 and/or cloud storage 1034.

FIG. 10A illustrates a schematic representation of an embodiment of a system comprising an electronic device with a function of determining a body core temperature of a person according to an embodiment of the disclosure.

Referring to FIG. 10A, it is shown that in an embodiment of the disclosure, the system 1000 may comprise the electronic device 1001, a remote server 1027 and/or cloud storage 1034 and has at least one communication channel 1030, 1031, 1032 for transferring data between the electronic device 1001 and remote server 1027 and/or cloud storage 1034.

Said at least one communication channel may be a direct (e.g., wired) communication channel or a wireless communication channel. For example, the wireless communication channel 1030 may be established between the communication module 213 of the electronic device 201, 1001 and an external device (e.g., device 1026), the wireless communication channel 1031 may be established between the communication module 213 of the electronic device 201, 1001 and the external device 1025, 1026, server 1027 or cloud storage 1034. The communication may be through a first network 1028 (e.g., a short-range communications network such as Bluetooth R, wireless communication (Wi-Fi), or infrared-range data transmission (IrDA)) or a second network 1029 (e.g., a network long distance communications, such as a cellular network, the Internet, or a computer network (e.g., a local area network or wide area network (WAN)).

In an embodiment of the system 1000 shown in FIG. 10A, the electronic device 201, 1001 uses at least a measurement data set collected from the sensors 205, 206, 207, 208 of the electronic device to determine the body core temperature of the user, T_{core}. Also, the electronic device 201, 1001 performs prediction of microclimate parameters around the electronic device, body morphology parameters and peripheral microcirculation parameters of the user. The prediction is based on applying a prediction algorithm trained as shown above by taking into account the profile data of other users (test subjects), measurement data sets pre-measured from other users, and reference data sets pre-measured by external reference measurement tools. In this embodiment of the disclosure, the prediction algorithm may be implemented as software 1033, including one or more instructions, which may be stored on a machine-readable storage medium (e.g., memory 212, 1012 of the electronic device 201, 1001) that is machine-readable, i.e., by the electronic device 201, 1001 and be executed by the at least one processor 203 of the electronic device 201, 1001. For example, the at least one processor 203 of the electronic device 201, 1001 may be programmed to cause at least one of one or more instructions stored in memory 212, 1012 and execute it with or without the use of one or more other components under the control of the at least one processor 203.

FIG. 10B illustrates a schematic representation of an embodiment of a system comprising an electronic device with a function of determining a body core temperature of a person according to an embodiment of the disclosure.

In an embodiment of the system 1000 shown in FIG. 10B, the prediction algorithm may be implemented in the software 1033, including one or more instructions, which may be stored on a computer-readable storage medium located on a remote server 1027. The at least one processor 203 of the electronic device 201, 1001 may be configured to enable the communication module 213 to run the prediction algorithm implemented in the software 1033 on the remote server 1027. In this embodiment of the disclosure, the at least one processor 203 of the electronic device 201, 1001 may enable the communication module 213 to transmit data to the remote server 1027 via communication channel 1032 and receiving data from the remote server 1027 via communication channel 1032, in particular, the at least one processor 203 can download at least a profile data of the user and a measurement data set collected from sensors 205, 206, 207, 208 of the electronic device from the memory 212, 1012 of the electronic device 201, 1001, and instruct the communication module 213 to transmit this data over the communication channel 1032 to the remote server 1027. Based on at least of a measurement data set collected from the sensors 205, 206, 207, 208 of the electronic device, the value of the body core temperature of the user, T_{core}, can be calculated by the software 1033, which can then be transmitted over the communication channel 1032 to the communication module 213, which, in turn, can transmit it to the at least one processor 203 of the electronic device 201, 1001. The at least one processor can store the value of the body core temperature, T_{core}, of the user received from the communication module 213 in the memory 212, 1012, and also output information about the body core temperature of the user to the output device 210 to inform the user.

In an embodiment of the disclosure, a function of controlling the body core temperature of the user from other electronic devices via a remote server and/or cloud storage may be implemented in the system 1000. For example, the user can turn on the body core temperature measurement mode from the device 1025, in which case the electronic device 201, 1001 (for example, a smart watch, a smart ring) can be instructed to determine the body core temperature of the user via the server 1027 or cloud storage 1034. In an embodiment of the disclosure, the turning on of the core temperature measurement mode can be requested by the attending physician or sports trainer, for example, from the device 1025.

In an embodiment of the disclosure, the remote server 1027 and/or cloud storage 1034 can be configured to create and store a user database, which may contain the following information: profile data of the user, measurement data sets, dates and times of collection of each of data measurement set, the values of the body core temperatures of the user which are determined by the device for each of measurement data set, as well as other necessary information. In particular, the user database can also store such data as: microclimate parameters around the electronic device, body morphology parameters of the user, peripheral microcirculation parameters of the user calculated during operation of the software 1033 that implements the prediction algorithm.

In an embodiment of the disclosure, the accessibility of the user database from different electronic devices (for example, a smartphone, etc.) that can synchronize this data with each other using cloud technologies can be implemented. For example, a profile data of the user (a gender, an age, a height and a weight) can be entered or adjusted by the user from the device 1025, and the electronic device 201, 1001 (for example, a smart watch, a smart ring) accesses this data through the server 1027 or cloud storage 1034. In an embodiment of the disclosure, the accessibility of a user database for a physician or sports trainer may be implemented.

In an embodiment of the disclosure, it is possible to use the data collected in the user database stored on the server 1027 or in the cloud storage 1034 to specify the prediction when determining the body core temperature of the user. For example, based on the data collected in the user database, the initially obtained body morphology parameters and peripheral microcirculation parameters of the user can be specified based on repeated measurements. Further, these specified body morphology parameters and peripheral microcirculation parameters of the user can be transmitted to the at least one processor 203 of the electronic device 201, 1001 and taken into account when predicting the body core temperature of the user along with data taken from the sensors of the electronic device.

In an embodiment of the disclosure, it is possible to use the data collected in the user database stored on the server 1027 or in the cloud storage 1034 to control the readings measured by the sensors of the electronic device. For example, based on this data, a deviation of said parameters from their average values can be determined, indicating, for example, a measurement error.

It should be noted that some embodiments of the disclosure are described with reference to various objects. However, one skilled in the art will appreciate from the foregoing and subsequent description that, unless otherwise indicated, in addition to any combination of features relating to the same type of subject matter, any combination of features relating to different subjects is also considered to be disclosed in this application. However, all of the features can be combined to provide synergistic effects that are more than just the summation of the features.

Although the disclosure has been illustrated in the drawings and described in detail in the foregoing description, these drawings and description are to be considered illustrative, and not restrictive. The disclosure is not limited to the disclosed embodiments of the disclosure. Those skilled in the art will be able to foresee and practice other variations of the disclosed embodiments of the disclosure as a result of studying the drawings, the disclosure, and the dependent claims.

In the claims, the word “comprising” does not exclude other elements or components, and mentioning of an element in the singular does not exclude a plurality of such elements. The mere fact that certain measures are mentioned in mutually different dependent claims does not mean that a combination of these measures cannot be used to advantage. Any reference designations in the claims should not be construed as limiting its scope.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device for determining a body core temperature of a user, the electronic device comprising:
at least one internal temperature sensor configured to measure an internal temperature inside the electronic device;
at least one skin temperature sensor configured to measure a skin surface temperature of the user;
at least one bioimpedance sensor configured to measure at least one body impedance parameter of the user;
at least one photoplethysmogram (PPG) sensor configured to measure at least two photoplethysmogram (PPG) signals with different wavelengths;
memory, comprising one or more storage media, storing instructions; and
at least one processor communicatively coupled to the at least one internal temperature sensor, the at least one skin temperature sensor, the at least one bioimpedance sensor, the at least one PPG sensor, and the memory,
wherein the instructions, when executed by the at least one processor individually or collectively, cause the electronic device to:
obtain the internal temperature inside the electronic device, the skin surface temperature of the user, the at least one body impedance parameter of the user and the at least two PPG signals with different wavelengths,
obtain a measurement data set including the internal temperature, the skin surface temperature, the at least one body impedance parameter of the user and the at least two PPG signals,
determine microclimate parameters around the electronic device based on a local ambient temperature and a local temperature,
determine body morphology parameters of the user based on profile data of the user and the at least one body impedance parameter of the user,
determine peripheral microcirculation parameters of the user body based on perfusion parameters of the user body which are calculated from the at least two PPG signals with different wavelengths, and
determine the body core temperature of the user based on at least one of the measurement data set, the microclimate parameters around the electronic device, the body morphology parameters of the user, or the peripheral microcirculation parameters of the user.

2. The electronic device of claim 1,
wherein the memory further stores the profile data of the user, and
wherein the instructions, when executed by the at least one processor individually or collectively, further cause the electronic device to:
determine the local ambient temperature around the electronic device based on the internal temperature inside the electronic device, and
determine the local temperature between the electronic device and the user skin based on the local ambient temperature and the skin surface temperature of the user.

3. The electronic device of claim 1, wherein the instructions, when executed by the at least one processor individually or collectively, further cause the electronic device to:
determine the microclimate parameters based on at least one sub-algorithm trained by using the profile data of other users, measurement data sets pre-measured from other users, and reference data sets pre-measured by at least one external reference measurement device,
determine the body morphology parameters based on the at least one sub-algorithm trained by using the profile data of other users, measurement data sets pre-measured from other users, and reference data sets pre-measured by the at least one external reference measurement device,
determine the peripheral microcirculation parameters based on the at least one sub-algorithm trained by using the profile data of other users, measurement data sets pre-measured from other users, and reference data sets pre-measured by the at least one external reference measurement device, and
determine the body core temperature based on the at least one sub-algorithm trained by using the profile data of other users, measurement data sets pre-measured from other users, and reference data sets pre-measured by the at least one external reference measurement device.

4. The electronic device of claim 1, wherein the at least one body impedance parameter of the user includes a magnitude of body impedance of the user, a phase angle of body impedance of the user, or a magnitude of a contact impedance.

5. The electronic device of claim 1, wherein the instructions, when executed by the at least one processor individually or collectively, further cause the electronic device to:
determine, based on the body morphology parameters of the user, an amount of a fat tissue, a muscle tissue, water in the user body, a ratio of extracellular and intracellular water in the user body and a condition of the user skin.

6. The electronic device of claim 1, wherein the instructions, when executed by the at least one processor individually or collectively, further cause the electronic device to:
filter the at least two PPG signals with different wavelengths from motion artifacts,
separate the at least two PPG signals with different wavelengths into time-constant and non-time-constant components of time series, and
transform the time series into a frequency domain or a frequency-time domain based on subsequent extraction of an amplitude-phase harmonic characteristics of the at least two PPG signals.

7. The electronic device of claim 6, wherein the instructions, when executed by the at least one processor individually or collectively, further cause the electronic device to:
transform the time series into the frequency domain or a frequency-time domain by using at least one of a Fourier transform, a Hilbert-Huang transform or a Wavelet transform.

8. A method performed by an electronic device for determining a body core temperature of a user, the method comprising:
obtaining, by the electronic device, an internal temperature inside the electronic device, a skin surface temperature of the user, at least one body impedance parameter of the user and at least two PPG signals with different wavelengths;
obtaining, by the electronic device, a measurement data set including the internal temperature, the skin surface temperature, the at least one body impedance parameter of the user and the at least two PPG signals;
determining, by the electronic device, microclimate parameters around the electronic device based on a local ambient temperature and a local temperature;
determining, by the electronic device, body morphology parameters of the user based on profile data of the user and the at least one body impedance parameter of the user;
determining, by the electronic device, peripheral microcirculation parameters of the user body based on perfusion parameters of the user body which are calculated from the at least two PPG signals with different wavelengths; and
determining, by the electronic device, the body core temperature of the user based on at least one of the measurement data set, the microclimate parameters around the electronic device, the body morphology parameters of the user, or the peripheral microcirculation parameters of the user.

9. The method of claim 8, wherein the determining microclimate parameters around the electronic device based on the internal temperature and the skin surface temperature, comprises:
determining the local ambient temperature around the electronic device based on the internal temperature inside the electronic device; and
determining the local temperature between the electronic device and the user skin based on the local ambient temperature and the skin surface temperature of the user.

10. The method of claim 8,
wherein the determining the microclimate parameters, comprises:
determining the microclimate parameters based on at least one sub-algorithm trained by using profile data of other users, measurement data sets pre-measured from other users, and reference data sets pre-measured by at least one external reference measurement device,
wherein the determining the body morphology parameters comprises:
determining the body morphology parameters based on the at least one sub-algorithm trained by using the profile data of other users, the measurement data sets pre-measured from other users, and the reference data sets pre-measured by the at least one external reference measurement device,
wherein the determining the peripheral microcirculation parameters comprises:
determining the peripheral microcirculation parameters based on the at least one sub-algorithm trained by using the profile data of other users, the measurement data sets pre-measured from other users, and the reference data sets pre-measured by the at least one external reference measurement device, and
wherein the determining the body core temperature, comprises:
determining the body core temperature, based on the at least one sub-algorithm trained by using the profile data of other users, the measurement data sets pre-measured from other users, and the reference data sets pre-measured by the at least one external reference measurement device.

11. The method of claim 8, wherein the at least one body impedance parameter of the user includes a magnitude of body impedance of the user, a phase angle of body impedance of the user, or a magnitude of a contact impedance.

12. The method of claim 8, wherein the determining the body core temperature of the user, comprises:
determining, based on the body morphology parameters of the user, an amount of a fat tissue, a muscle tissue, water in the user body, a ratio of extracellular and intracellular water in the user body and a condition of the user skin.

13. The method of claim 8, wherein the determining peripheral microcirculation parameters of the user body, comprises:
filtering the at least two PPG signals with different wavelengths from motion artifacts;
separating the at least two PPG signals with different wavelengths into time-constant and non-time-constant components of time series; and
transforming the time series into a frequency domain or a frequency-time domain based on subsequent extraction of an amplitude-phase harmonic characteristics of the at least two PPG signals.

14. The method of claim 13, wherein the transforming the time series, comprises:
transforming the time series into the frequency domain or a frequency-time domain by using at least one of a Fourier transform, a Hilbert-Huang transform or a Wavelet transform.

15. One or more non-transitory computer-readable storage media storing one or more computer programs including computer-executable instructions that, when executed by at least one processor of an electronic device individually or collectively, cause the electronic device to perform operations, the operations comprising:
obtaining, by the electronic device, an internal temperature inside the electronic device, a skin surface temperature of a user, at least one body impedance parameter of the user and at least two PPG signals with different wavelengths;
obtaining, by the electronic device, a measurement data set including the internal temperature, the skin surface temperature, the at least one body impedance parameter of the user and the at least two PPG signals;
determining, by the electronic device, microclimate parameters around the electronic device based on a local ambient temperature and a local temperature;
determining, by the electronic device, body morphology parameters of the user based on profile data of the user and the at least one body impedance parameter of the user;
determining, by the electronic device, peripheral microcirculation parameters of the user body based on perfusion parameters of the user body which are calculated from the at least two PPG signals with different wavelengths; and
determining, by the electronic device, body core temperature of the user based on at least one of the measurement data set, the microclimate parameters around the electronic device, the body morphology parameters of the user, or the peripheral microcirculation parameters of the user.

16. The one or more non-transitory computer-readable storage media of claim 15, the operations further comprising:
determining the local ambient temperature around the electronic device based on the internal temperature inside the electronic device; and
determining the local temperature between the electronic device and the user skin based on the local ambient temperature and the skin surface temperature of the user.
